(19) United States
(12) Patent Application Publication
(10) Pub. No.: US 2003/0224477 A1
(43) Pub. Date: Dec. 4, 2003

(54) Optimized promoter constructs

(76) Inventors: Michael W. Heartlein, Boxborough, MA (US); Justin Chace Lamsa, Westminster, MA (US); Douglas A. Treco, Arlington, MA (US); Richard F. Selden, Wellesley, MA (US); Michael F. Concino, Bolton, MA (US); Heidi Kempinski, Charlton, MA (US)

Correspondence Address: FISH & RICHARDSON PC, 225 FRANKLIN ST, BOSTON, MA, 02110, US

(21) Appl. No.: 10/160,851

(22) Filed: May 31, 2002

(51) Int. Cl. C07H 21/04; C12P 21/02; C12N 5/08; C12N 9/24

(52) U.S. Cl. 435/69.1; 435/320.1; 435/366; 435/200; 530/383; 530/384; 536/23.2

(57) ABSTRACT

The invention features constructs and related methods for expression of products in mammalian cells, e.g., human cells. Constructs include a human γ-actin, β-actin, fibronectin, YY1, or β-tubulin promoter region operably linked to a heterologous nucleic acid sequence.

### BACKGROUND

Regulation of eukaryotic gene expression is in large part controlled by the action of transcriptional regulatory elements. Transcriptional regulatory regions include promoters, enhancers, repressors, insulating regions (e.g., matrix attachment regions), binding sites for individual transcription factors, and composite elements (closely located, e.g., adjacent or even overlapping, transcription factor binding sites that function cooperatively). These regulatory sequences are predominantly located upstream (5′) of the transcription initiation site (the cap site) in naturally occurring genes, although some elements can occur within a gene, flanking a gene, or downstream (3′) of the cap site, e.g., in the 5′ untranslated sequence region (5′ UTS). The number and type of regulatory elements varies with each naturally occurring gene. A striking feature of many transcriptional regulatory elements is their modular structure.

### SUMMARY

The invention is based, in part, on the inventors' discovery and development of constructs, e.g., plasmid constructs, for expression, particularly long term expression, of a nucleic acid sequence, e.g., an exogenous nucleic acid sequence, in a cell, e.g., a human cell, preferably a human fibroblast. The constructs described herein include the use of a promoter region sequence from one or more of: a γ-actin gene, a fibronectin gene, a β-tubulin gene, a YY1 gene, a β-actin gene. In preferred embodiments, the constructs described herein include all or part of the promoter region sequence shown as SEQ ID NO: 1 (a human γ-actin promoter region), SEQ ID NO:2 (a human fibronectin promoter region), SEQ ID NO:3 (a human β-tubulin promoter region), or SEQ ID NO:4 (a human YY1 promoter region). For example, the constructs described herein can include at least 2 contiguous nucleotides, preferably at least 100, 500, 1000, 1500, 2000, 3000 contiguous nucleotides from the promoter region sequence shown as SEQ ID NO:1, SEQ ID NO:2, SEQ ID NO:3, or SEQ ID NO:4. In preferred embodiments, a construct described herein includes one or more of the following regulatory elements: an enhancer; a 5′ UTS, e.g., a heterologous 5′ UTS or a 5′ UTS fusion, e.g., including an intron; a MAR; a U1 intron. In preferred embodiments, the constructs described herein include two or more regulatory elements fused to each other.

“Long term expression” of a nucleic acid sequence refers to the presence (in vitro or in vivo) of detectable amounts of a polypeptide encoded by the nucleic acid sequence for at least 4 weeks, preferably at least 6 weeks, more preferably at least 10, 20, 30, 40, 50, 60, 70, 80, 90, 100 weeks or more. Various methods known in the art can be used to assay for detectable amounts of a polypeptide, including immunodetection of the polypeptide, e.g., an ELISA assay, e.g., an ELISA assay described herein; assaying for an activity of the polypeptide; or purification of the polypeptide.

Accordingly, in one aspect the invention features an isolated nucleic acid comprising a promoter region sequence from one or more of: a γ-actin gene, a fibronectin gene, a β-tubulin gene, a YY1 gene, a β-actin gene. Preferably, the isolated nucleic acid comprises the nucleotide sequence of any of: SEQ ID NO:1, SEQ ID NO:2, SEQ ID NO:3, SEQ ID NO:4, or a functional fragment thereof. Preferably, a fragment is at least 100, 200, 300, 400, 500, 600, 700, 800, 900, 1000, 1200, 1500, 1700, 2000, 2500, 3000, 3500, 4000, 4500, 5000, or more nucleotides.

In a preferred embodiment, the isolated nucleic acid comprises a fragment of the nucleotide sequence of SEQ ID NO: 1, SEQ ID NO:2, SEQ ID NO:3, or SEQ ID NO:4. In preferred embodiments, the fragment is at least 2 nucleotides in length. Preferably, the fragment is between 500 and 8000 nucleotides in length. More preferably, the fragment is between 500 and 7000 nucleotides in length. Even m ore preferably, the fragment is between 1000 and 4000 nucleotides in length. In a preferred embodiment, the fragment has at least 10%, preferably at least 20%, 30%, 40%, 50%, 50%, 70%, 80%, or more, of a regulatory activity, e.g., the ability to promote or enhance transcription, of the sequence of SEQ ID NO: 1, SEQ ID NO:2, SEQ ID NO:3, or SEQ ID NO:4.

In a preferred embodiment, the isolated nucleic acid further includes vector, e.g., plasmid, nucleic acid sequence.

In a preferred embodiment, the isolated nucleic acid sequence includes a nucleic acid sequence encoding a heterologous polypeptide.

In another aspect, the invention features an isolated nucleic acid comprising a nucleotide sequence which is at least 60%, preferably 70%, 80%, 90%, 95%, 98%, 99%, or more, identical to the nucleotide sequence of SEQ ID NO: 1, SEQ ID NO:2, SEQ ID NO:3, or SEQ ID NO:4 or a fragment thereof.

In a preferred embodiment, the isolated nucleic acid further includes plasmid nucleic acid sequence.

In a preferred embodiment, the isolated nucleic acid sequence includes a nucleic acid sequence encoding a heterologous polypeptide.

In another aspect, the invention features a host cell which contains an isolated nucleic acid sequence described herein.

In a preferred embodiment, the host cell is a mammalian cell.

In a preferred embodiment, the host cell is a human cell.

In a preferred embodiment, the host cell is a human fibroblast.

In another aspect, the invention features a construct, e.g., a plasmid construct, suitable for expression in a mammalian cell, e.g., a human cell, preferably a human fibroblast, comprising a human γ-actin promoter region operably linked to a heterologous nucleic acid sequence.

In a preferred embodiment, the construct includes at least one, preferably two, more preferably three, or more, of the following elements: a) an enhancer, preferably a CMV enhancer, preferably placed at about cap −300 bp to cap −1100 bp, more preferably placed at about cap −825; b) a heterologous 5′ UTS, preferably an aldolase 5′ UTS, an EF1-α 5′ UTS, or a β-actin 5′ UTS; c) a γ-actin 5′ UTS fusion, preferably of cap +25 to cap +50 bp in length; or d) a MAR, preferably a β interferon (βI) MAR.

In a preferred embodiment, the construct includes a CMV enhancer at about cap −825 bp, and a γ-actin 5′ UTS fusion of cap +25 base pairs in length fused to an aldolase 5′ UTS.

In a preferred embodiment, the γ-actin promoter region includes at least 2, preferably 100, 250, 500, 1000, 2000, 3000, 5000, 6000, or 6500, contiguous nucleotides from the sequence of SEQ ID NO: 1.

In a preferred embodiment, the γ-actin promoter region is between 0.9 kb and 7.2 kb in length, preferably between 3.6 kb and 6.5 kb in length.

In a preferred embodiment, the promoter region includes less than the full length promoter region.

In a preferred embodiment, the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase) β-galactosidase, N-acetylgalactosamine-4-sulfatase (arylsulfatase B), β-glucuronidase or biologically active fragment thereof.

In another aspect, the invention features a construct, e.g., a plasmid construct, suitable for expression in a mammalian cell, e.g., a human cell, preferably a human fibroblast, comprising a human β-tubulin promoter region operably linked to a heterologous nucleic acid sequence.

In a preferred embodiment, the construct further includes at least one, preferably two, more preferably three, of the following elements: a) a heterologous 5′ UTS, preferably an aldolase 5′ UTS, an EF1-α 5′ UTS, or a β-actin 5′ UTS; b) an enhancer, preferably a CMV enhancer; or c) a U1 intron, preferably a GAPDH U1 intron or a β-actin U1 intron.

In a particularly preferred embodiment, the construct includes an EF1-α 5′ UTS.

In a preferred embodiment, the construct includes at least two contiguous nucleotides from the sequence of SEQ ID NO:3. Preferably, the construct includes at least 100, 200, 500, 1000, 1500, 2000, or 2500, contiguous nucleotides from the sequence of SEQ ID NO:3

In a preferred embodiment, the β-tubulin promoter region is between 0.6 kb and 12 kb in length, preferably between 0.6 kb and 2.3 kb in length.

In a preferred embodiment, the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or biologically active fragment thereof.

In a preferred embodiment, the promoter region includes less than the full length promoter region.

In another aspect, the invention features a construct, e.g., a plasmid construct, suitable for expression in a mammalian cell, preferably a human cell, preferably a human fibroblast, comprising a human YY1 promoter region operably linked to a heterologous nucleic acid.

In a preferred embodiment, the construct includes at least one, preferably two, more preferably three, of the following elements: a) an enhancer, preferably a CMV enhancer, preferably placed between about cap −100 to cap −1000 base pairs, preferably about cap −860 base pairs; b) at least one heterologous 5′ UTS, preferably an aldolase 5′ UTS, EF1-α 5′ UTS or a β-actin 5′ UTS; or c) a matrix attachment region (MAR), preferably a beta-interferon MAR.

In a particularly preferred embodiment, the construct includes: a) a CMV enhancer placed at about cap −860 bp; b) an EF1-α 5′ UTS fused to a β-actin 5′ UTS; and c) a βI MAR In a preferred embodiment, the construct includes at least two, preferably at least 100, 200, 500, 1000, 1500, 2000, 2500, 3000 contiguous nucleotides from the sequence of SEQ ID NO:4

In a preferred embodiment, the YY1 promoter region is between 1.0 kb and 3.1 kb, preferably between 2 kb and 3.1 kb, in length.

In a preferred embodiment, the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or a biologically active fragment thereof.

In a preferred embodiment, the promoter region includes less than the full length promoter region.

In another aspect, the invention features a construct, e.g., a plasmid construct, suitable for expression in a mammalian cell, preferably a human cell, e.g., a human fibroblast, comprising a human fibronectin promoter region operably linked to a heterologous nucleic acid sequence. Preferably, the fibronectin promoter region promoter region includes at least two contiguous nucleotides, preferably at least 100, 200, 500, 550, 600, 1000, 1500, 2000, 3000, 4000, 5000, 6000, 7000, or more nucleotides from the sequence shown as SEQ ID NO:2.

In a preferred embodiment, the construct includes at least one, preferably two, more preferably three or more, of the following elements: a) an enhancer, preferably a CMV enhancer, preferably placed between about cap −50 to cap −700 bp; b) a MAR, preferably a βI MAR; c) a U1 intron, preferably an EF1-A, aldolase, or GAPDH U1 intron; d) a 5′ UTS, preferably a β-actin, aldolase, or EF1-α 5′ UTS; d) a fibronectin 5′ UTS fusion, preferably between about cap +147 to cap +270 bp in length.

In a preferred embodiment, the construct includes a 5′ UTS, e.g., a β-actin 5′ UTS, fused to a U1 intron, e.g., an EF1-α U1 intron, and at least one, preferably two, of the following elements: an enhancer, e.g., a CMV enhancer; a MAR, e.g., a βI MAR; a fibronectin 5′ UTS fusion, preferably of between about cap +147 to cap +270 bp in length.

In a preferred embodiment, the human fibronectin promoter region is between 0.5 kb and 8.8 kb in length, preferably between 2 kb and 6 kb, more preferably between 3 kb and 5 kb.

In a preferred embodiment, the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or a biologically active fragment thereof. In a preferred embodiment, the promoter region includes less than the full length promoter region.

In another aspect, the invention features a construct, e.g., a plasmid construct, suitable for expression in a mammalian cell, e.g., a human cell, preferably a fibroblast, which includes a human fibronectin promoter region operably linked to a heterologous nucleic acid sequence, provided that said promoter region includes at least one of the following elements: a) an enhancer; b) a U1 intron.

In another aspect, the invention features a construct, e.g., a plasmid construct, suitable for expression in a mammalian cell, e.g., a human cell, preferably a fibroblast, which includes a human β-actin promoter region operably linked to a heterologous nucleic acid sequence, provided that the β-actin promoter region includes at least one of the following elements: a) an MAR; b) a β-actin 5′ flank addition; or c) a β-actin 5′ UTS fusion.

In a preferred embodiment, the promoter region includes less than the full length promoter region.

In another aspect, the invention features a construct suitable for expression in a mammalian cell, e.g., a human cell, preferably a human fibroblast, which includes a human β-actin promoter region operably linked to a heterologous nucleic acid sequence, where the construct includes at least one, preferably two, more preferably three or more, of the following elements: a) MAR, preferably a β-interferon (βI) MAR; b) an enhancer, preferably a CMV enhancer or a CMV based enhancer (e.g., a 4 tandem 53 base pair repeat element derived from a CMV enhancer, preferably placed between about cap −50 and cap −500 bp); c) a heterologous 5′ UTS, preferably an aldolase 5′ UTS; d) a 5′ flank addition; e) a U1 intron, preferably an EF1-α U1 intron; f) a β-actin 5′ UTS fusion, preferably of about cap +20 to cap +120 base pairs in length, more preferably of about cap +50 to cap +100 base pairs in length, or about cap +77 base pairs in length. Preferably, if the construct includes an enhancer, it does not include a heterologous 5′ UTS and if the construct includes a heterologous 5′ UTS, it does not include an enhancer.

In a preferred embodiment, the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or a biologically active fragment thereof.

In a preferred embodiment, the promoter region includes less than the full length promoter region.

In another aspect, the invention features a cell, preferably a mammalian cell, preferably a human cell, e.g., a fibroblast, transfected with a construct described herein.

In another aspect, the invention features a method of producing a substance. The method includes a) providing a mammalian cell, e.g., a human cell, preferably a fibroblast, which includes a construct described herein; b) allowing the cell to express a heterologous polypeptide, e.g., Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or a biologically active fragment thereof; and optionally c) isolating the polypeptide from the cell or its culture media.

In a preferred embodiment, the polypeptide is expressed in vitro.

In a preferred embodiment, the polypeptide is expressed in vivo.

In a preferred embodiment, the cell is an autologous cell. In another preferred embodiment, the cell is an allogeneic cell. In yet another preferred embodiment, the cell is a xenogeneic cell.

The invention also features a polypeptide, e.g., a therapeutic polypeptide, produced by a method described herein. In one embodiment, the method includes: a) providing a mammalian cell, e.g., a human cell, preferably a fibroblast, which includes a construct described herein which includes a promoter region operably linked to a sequence encoding a heterologous polypeptide (e.g., Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or a biologically active fragment thereof); b) allowing the cell to express the polypeptide; and c) isolating the polypeptide from the cell.

In another aspect, the invention features a method of supplying a substance to a subject, e.g., a mammal, e.g., a human. The method includes (a) providing a mammalian cell, preferably a human cell, preferably a fibroblast, which includes a construct described herein; (b) allowing the cell to express a heterologous polypeptide, e.g., Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or a biologically active fragment thereof; and (c) administering the heterologous polypeptide to the subject.

In a preferred embodiment, the polypeptide is expressed in vitro.

In a preferred embodiment, the polypeptide is expressed in vivo.

In a preferred embodiment, the cell is an autologous cell. In another preferred embodiment, the cell is an allogeneic cell. In yet another preferred embodiment, the cell is a xenogeneic cell.

In another aspect, the invention features a method of treating a disorder in a subject, e.g., a mammal, e.g., a human. The method includes a) providing a mammalian cell, e.g., a human cell, preferably a fibroblast, which includes a construct described herein; and b) allowing the cell to express a heterologous polypeptide in vivo in the subject. Preferably, the polypeptide is e.g., Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or a biologically active fragment thereof.

In a preferred embodiment, the polypeptide is expressed in vitro.

In a preferred embodiment, the polypeptide is expressed in vivo.

In a preferred embodiment, the cell is an autologous cell. In another preferred embodiment, the cell is an allogeneic cell. In yet another preferred embodiment, the cell is a xenogeneic cell.

In another aspect, the invention features a method of providing a heterologous protein to a subject, e.g., a mammal, e.g., a human. The method includes: (a) providing a mammalian cell, e.g., a human cell, preferably a fibroblast, which includes a construct described herein; and (b) allowing the cell to produce the heterologous protein in said subject.

Preferably, the polypeptide is e.g., Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or a biologically active fragment thereof.

In a preferred embodiment, the polypeptide is expressed in vitro.

In a preferred embodiment, the polypeptide is expressed in vivo.

In a preferred embodiment, the cell is an autologous cell. In another preferred embodiment, the cell is an allogeneic cell. In yet another preferred embodiment, the cell is a xenogeneic cell.

In another aspect, the invention features a method of treating a disorder in a subject, e.g., a mammal, e.g., a human. The method includes: a) identifying a subject in need of a product; and b) introducing into the subject a construct described herein, wherein the construct causes the production of the product in an amount sufficient to ameliorate a symptom of the disorder.

Preferably, the product is e.g., Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or a biologically active fragment thereof.

In a preferred embodiment, the product is expressed in vitro.

In a preferred embodiment, the product is expressed in vivo.

In a preferred embodiment, the cell is an autologous cell. In another preferred embodiment, the cell is an allogeneic cell. In yet another preferred embodiment, the cell is a xenogeneic cell.

In a preferred embodiment, the product is Factor VIII and the method further comprises evaluating the level of Factor VIII in a blood sample of the subject. A blood sample can be, e.g., whole blood, blood cells, serum or plasma. The evaluating step includes: (a) contacting a blood sample of the subject with a first antibody to Factor VIII, thus forming a reaction mixture; (b) contacting the reaction mixture, preferably after performing step (a), with a labeled second antibody to Factor VIII; and (c) detecting the amount of label in the reaction mixture. The amount of label is correlated to the amount of Factor VIII in the sample.

In a preferred embodiment, the first antibody is coated onto a receptacle, e.g., a multiple well plate, and the sample is added to the antibody-coated receptacle to form a reaction mixture. A preferred sample is plasma.

In a preferred embodiment, less than 6.25 mU/mL, preferably less than 6 mU/mL, 5.5 mU/mL, 5 mU/mL, 4.5 mU/mL, 4 mU/mL, or 3.5 mU/mL of Factor VIII can be detected. While not wanting to be bound by theory, it is believed that the high sensitivity of the assay is due to any one or more of: (a) using a first antibody concentration of less than 10, preferably less than 9, more preferably less than 8, 7, 6, 5, 4, 3, 2 or 1 μg/mL; (b) a low assay volume (e.g., less than 100 μL of sample, preferably less than 80, 60, or about 50 μL of sample); (c) performing two distinct steps for first and second antibody reactions; (d) incubating the reaction at higher than 4° C., preferably between 5 and 35° C.; more preferably between about 10 to 30° C., even more preferably at or about room temperature; (e) having between about 5 to 40%, preferably between 10 to 30%, more preferably about 20% plasma in the reaction mixture. For example, non cross-reacting, Factor VIII-free plasma, e.g., plasma from hemophiliac subjects, can be added to maintain 20% plasma in the reaction mixtures.

In another aspect, the invention features a method of evaluating the level of Factor VIII in a sample, e.g., a blood sample of the subject. A blood sample can include, e.g., whole blood, cells, serum or plasma. The method includes: (a) contacting a blood sample of the subject with a first antibody to Factor VIII, thus forming a reaction mixture, preferably in the absence of a secondary antibody; (b) preferably thereafter, contacting the reaction mixture with a second labeled antibody to Factor VIII; and (c) detecting the amount of label in the reaction mixture. The amount of label is correlated to the amount of Factor VIII in the sample.

A blood sample can be, e.g., whole blood, blood cells, serum, or plasma. A preferred sample is plasma, e.g., human plasma.

In a preferred embodiment, the first antibody is coated onto a receptacle, e.g., a multiple well plate, and the sample is added to the antibody-coated receptacle to form the reaction mixture. A preferred coating concentration is less than 10 μg/mL, preferably less than 8, 6, 5, 4, 3 or 2 μg/mL.

In a preferred embodiment, less than 6.25 mU/mL of Factor VIII, e.g., in plasma or serum, can be detected by the method. More preferably, less than 5 mU/mL of Factor VIII can be detected. Even more preferably, less than 4 mU/mL, 3.5 mU/mL, or about 3 mU/mL of Factor VIII can be detected.

In a preferred embodiment, the first antibody is ESH-8. Preferably, ESH-8 is used (e.g., coated on a receptacle) at a concentration of less than 10 μg/mL, preferably less than 8, 6, 5, 4, 3 or 2 μg/mL. In one preferred embodiment, the coating concentration of ESH-8 is about 1 μg/mL.

In a preferred embodiment, the blood sample is less than 100 μL, preferably less than 80, 60, or about 50 μL.

In a preferred embodiment, the second labeled antibody is labeled ESH-4. The label is a detectable label, e.g., an detectable enzyme label, e.g., horseradish peroxidase, or an otherwise detectable label, e.g., a fluorescent, luminescent, or chemiluminescent label known in the art.

In another aspect, the invention features a method of evaluating the level of Factor VIII in a sample, e.g., a blood sample of the subject. A blood sample can include, e.g., whole blood, cells, serum or plasma. The method includes: (a) incubating a blood sample of the subject with a first antibody to Factor VIII to form a reaction mixture; (b) incubating the reaction mixture with a second labeled antibody to Factor VIII; and (c) detecting the amount of label in the reaction mixture. The method also includes one or more of the following limitations: (a) two distinct steps are performed for the first and second antibody incubations; (b) a low sample volume is used (e.g., less than 100 μL of sample, preferably less than 80, 60, or about 50 μL of sample, e.g., serum sample); (c) the first antibody is coated onto a receptacle at a concentration of less than 10 μg/mL, preferably less than 9 μg/mL, more preferably less than 8, 7, 6, 5, 4, 3, 2 or about 1 μg/mL; (d) the reaction mixture is incubated at a temperature higher than 4° C., preferably between 5° and 35° C.; more preferably between about 10° to 30° C., even more preferably at or about room temperature; (e) the reaction mixture includes between about 5 to 40%, preferably between 10 to 30%, more preferably about 20% plasma, e.g., non cross-reacting, Factor VIII-free plasma, e.g., plasma from hemophiliac subjects; (f) a wash step is performed after the first and/or second antibody incubation step, preferably using a chelating agent, e.g., EDTA, in the wash buffer. The method has a sensitivity for Factor VIII of at least about 6 mU/mL, preferably at least about 5 mU/mL, more preferably at least about 4 mU/mL, most preferable at least about 3 mU/mL, e.g., 3.125 mU/mL.

In a preferred embodiment, the blood sample can be, e.g., whole blood, blood cells, serum, or plasma. Preferably, the sample is plasma.

In a preferred embodiment, the first antibody is ESH-8.

In a preferred embodiment, the second labeled antibody is labeled ESH-4. The label is a detectable label, e.g., an detectable enzyme label, e.g., horseradish peroxidase, or an otherwise detectable label, e.g., a fluorescent, luminescent, or chemiluminescent label known in the art.

In another aspect, the invention features a kit for the detection of Factor VIII. The kit includes (a) a first antibody to Factor VIII; (b) a second, preferably labeled, antibody to Factor VIII; and (c) instructions for using the first and second antibodies to detect at least 6 mU/mL, preferably at least 5 mU/mL, 4 mU/mL, or about 3 mU/mL Factor VIII, e.g., about 3.125 mU/mL Factor VIII. The first antibody is preferably ESH-8 and the second, labeled antibody is preferably labeled ESH-4. The label on the second antibody is a detectable label, e.g., an detectable enzyme label, e.g., horseradish peroxidase, or an otherwise detectable label, e.g., a fluorescent, luminescent, or chemiluminescent label known in the art.

In a preferred embodiment, the instructions comprise instructions to do one or more of the following: (a) perform two distinct steps for first and second antibody reactions; (b) use a low assay volume (e.g., less than 100 μL of sample, preferably less than 80, 60, or about 50 μL of sample, e.g., blood sample); (c) coat the first antibody onto a support, e.g., a receptacle such as a well, at a concentration of less than 10 μg/mL, preferably less than 9 μg/mL, more preferably less than 8, 7, 6, 5, 4, 3, 2 or about 1 μg/mL; (d) incubate the reaction at a temperature higher than 4° C., preferably between 5° and 35° C.; more preferably between about 10° to 30° C., even more preferably at or about room temperature; (e) include between about 5 to 40%, preferably between 10 to 30%, more preferably about 20% plasma, e.g., non cross-reacting, Factor VIII-free plasma, e.g., plasma from hemophiliac subjects, in the reaction mixture; (f) perform a washing step after each antibody reaction step, preferably using a chelating agent, e.g., EDTA, in the wash buffer.

A “construct” is defined herein as a nucleic acid molecule that has been modified to contain segments of nucleic acid that are combined and juxtaposed in a manner that would not otherwise exist in nature. The term encompasses plasmid and viral-based constructs.

A “promoter region” as used herein, refers to nucleotide sequence upstream of the transcription initiation site of a gene. A promoter region can encompass up to 15 kb or more upstream of the transcription initiation site, or a portion thereof. A promoter region may include, e.g., transcriptional regulatory elements. Preferably, a promoter region of a construct described herein includes at least 2 nucleotides, but less than the full length sequence, of the sequence shown as SEQ ID NO:1 SEQ ID NO:2, SEQ ID NO:3, or SEQ ID NO:4. As used herein, a “promoter region” can include the sequence of the basal promoter of a gene (the region of DNA to which RNA polymerase binds before initiating the transcription) and/or can include 5′ flank sequence upstream of the basal promoter. The basal promoter typically contains CCAAT-box and TATA-box sequence motifs. The CCAAT-box (consensus GGT/CCAATCT) typically resides 50 to 130 bases upstream of the transcriptional start site in a naturally occurring gene. The TATA-box typically resides 20 to 30 bases upstream of the transcriptional start site of a naturally occurring gene. Numerous proteins identified as TFIIA, B, or C, have been observed to interact with the TATA-box.

“Cap site” is used herein interchangeably with “transcription initiation site.” The nucleotide at which transcription starts is designated +1 (e.g., cap +1) and nucleotides are numbered from this reference point with negative numbers indicating upstream nucleotides and positive numbers indicating downstream nucleotides.

An “enhancer” element, as used herein, is a sequence whose presence increases gene transcription, e.g., increases RNA polymerase binding to a promoter, thereby initiating transcription. An enhancer is usually insufficient to cause transcription alone, but can typically assist any promoter placed in its vicinity. Enhancers can function at various distances from a promoter and in either orientation. For example, an enhancer can be placed 500, 1000, 2000, 3000 or more nucleotides from a promoter, and still increase expression. Enhancers are also orientation independent, i.e., the enhancer can be inverted without losing is function. Preferred enhancers used in the constructs described herein include the CMV enhancer (e.g., as described in Foecking & Hofstetter, 1986, Gene 45:101-105 and Meier & Stinski, 1996, Intervirology 39:331-42); SV40 enhancer (see, e.g., Banerji et al., 1981, Cell 27:299); polyoma virus enhancer (see, e.g., de Villiers & Schaffner, 1981, Nucleic Acids Res. 9:6251 and Veldman et al., 1985, Mol. Cell Biol. 5:649-658); human collagen alpha2(I) enhancer (see, e.g., Ihn et al., 1996, J. Biol. Chem. 271:26717-26723); immunoglobulin enhancer (see, e.g., Banerji et al., 1983, Cell 33:729-740); fibronectin enhancer (see, e.g., Spom & Schwarzbauer, 1995, Nucleic Acids Res. 23:3335-3342). The term enhancer is also intended to encompass an enhancer element derived from a naturally occurring enhancer element, e.g., a repeat element derived from the naturally occurring CMV enhancer sequence.

“5′ untranslated sequence” or “5′ UTS” or “5′ UT” refers to the untranslated nucleotide sequence present between the cap site and the initiation of translation. A 5′ UTS sequence can contain, e.g., a regulatory element and/or an intron. A 5′ UTS can be a heterologous 5′ UTS, i.e., a 5′ UTS from a gene other than the gene from which the promoter region sequence of a construct is derived, or it can be a “5′ UTS fusion,” i.e., a 5′ UTS derived from the same gene as that from which the promoter region sequence of a construct is derived. Preferred 5′ UTS includes 5′ UTS from mammalian genes, e.g., from human genes, although non-mammalian 5′ UTS can also be used. Preferred genes that are the source of 5′ UTS in the constructs described herein include an aldolase gene, an EF1-α gene, a β-actin gene, a γ-actin gene, a GAPDH gene. The nucleotide sequence of 5′ UTS regions can be found, e.g., by searching a database of known gene sequences for 5′ UTS sequence of a particular gene, e.g., by searching the online GenBank database of gene sequences, e.g., by using the gene name as a keyword.

As used herein, a “U1 intron” or “U1I” is a sequence element that contains only the spliced region of an intron, without any surrounding untranslated sequence (see Korb & Johnson, 1993, Nucleic Acids Res. 21(25):5901-8). A U1 intron element starts with a portion of a splice donor (SD) sequence, which has been modified to match a consensus SD sequence, and ends with a splice acceptor (SA) sequence that has been modified to match a consensus SA sequence. Preferred U1 introns used in the constructs and methods described herein are part of the 5′ UTS region of a gene, preferably a mammalian gene, more preferably a human gene, although non-mammalian U1 introns can also be used. Preferred U1 introns include an EF1-α U1 intron, which contains several Sp1 and Ap1 elements which seem to have additive effects on its promoter activity (Wakabayashi-Ito et al. 1994, J. Biol Chem 269:29831-7); a GAPDH U1 intron (Ercolani et al., 1988, J. Biol. Chem. 263 :15335-15341), a β-actin U1 intron (Nakajima-Iijima et al., 1985, Proc. Natl. Acad. Sci. U.S.A. 82:6133-6137; GenBank Accession No. M10277).

A “matrix attachment region” or “MAR” is a sequence element that can mediate the attachment of specific areas of interphase nuclear chromatin to the lamina of the nuclear matrix. The higher order structure of eukaryotic chromosomes consists of independent loop domains, which are thought to be separated from each other by the periodic attachment of MARs onto the nuclear matrix. MAR's can thereby serve as insulators of a transcription unit in a naturally occurring gene. The general attributes of MARs have been summarized in Boulikas, 1993, J. Cell Biochem. 52:14-22 and are reviewed in Allen et al., 2000, Plant Mol. Biol. 43:361-76). MARs often include potential origins of replication, relatively long A-T-rich stretches having, e.g., topoisomerase II binding sites and/or palindromic sequences. Some classes of MARs contain CT-rich stretches or may be enriched in TG-motifs. In addition, MAR's can include transcription factor binding sites and can contain potentially curved or kinked DNA. A construct described herein can include at least one MAR. Where more than one, e.g., two, MAR's are employed in a construct described herein, e.g., flanking 5′ and 3′ of a nucleic acid encoding a polypeptide, they may be the same or different. Preferred MARs described herein are mammalian MAR's, preferably human MAR's, although non-mammalian MAR's can also be used if they function in a mammalian cell. A preferred MAR is human β-interferon MAR (βI MAR), as described in, e.g., Bode et al. (1988) Biochemistry 27:4706-4711, which is hereby incorporated by reference in its entirety. Other MAR's that can be used in the constructs described herein include the keratin 18 (K18) MAR's (U.S. Pat. No. 5,840,555; Neznanov et al., 1993, Mol. Cell Biol. 13:2214-2223); chicken lysozyme 5′ MAR, which is known to function in mammalian cells (Phi-Ban et al., 1990, Mol. Cell Biol. 10:2302-2307); human β-globin 5′ MAR (Yu et al., 1994, Gene 139:139-145); MAR from T cell receptor beta; and human chromosome 19 MAR's, e.g., GenBank Accession Numbers Z35279, Z35288, Z35291, Z35290, Z35220, Z35221, Z35222, Z35223, and Z35224 (Nikolaev et al., 1996, Nuc. Acids Res. 24:1330-1336).

As used herein, a sequence that is “heterologous” to a subject sequence is a sequence that is not normally operably linked to the subject sequence in nature.

All publications cited in this application are incorporated herein by reference in their entirety. The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-C shows the nucleotide sequence of a human γ-actin promoter region from nucleotide −473 to nucleotide −7222 (SEQ ID NO: 1) of the promoter region. This sequence was identified by screening a human genomic leukocyte library using a probe derived from a known γ-actin promoter region sequence (GenBank Accession No. M19283). FIG. 1D shows a diagram of the human γ-actin promoter region

FIGS. 2A-D shows the nucleotide sequence of a human fibronectin promoter region (SEQ ID NO:2). This sequence was identified by screening a human genomic circulating whole blood library using a probe derived from known fibronectin promoter region sequence (GenBank Accession No. M26179 and/or M15801). FIG. 2E shows a diagram of the human fibronectin promoter region.

FIGS. 3A-E shows the nucleotide sequence of a human β-tubulin promoter region (SEQ ID NO:3). This sequence was identified by screening a human genomic placental library using a probe derived from known β-tubulin promoter region sequence (GenBank Accession No. X02344). FIG. 3F shows a diagram of the human β-tubulin promoter region.

FIG. 4A shows the nucleotide sequence of a human YY1 promoter region (SEQ ID NO:4). This sequence was identified by screening a human genomic leukocyte library using a probe derived from known YY1 5′ UTS region (GenBank Accession No. M77698, Z14077, and/or AF047455). FIG. 4B shows a diagram of the human YY1 promoter region.

FIG. 5A shows a plasmid map for a γ-actin promoter region-based construct, pXF8.941. The following elements are indicated on the construct. Human γ-actin promoter; CMV enhancer at about cap −825, aldolase 5′ UTS (including the intron from this region); the synthetic beta domain-deleted Factor VIII (hFVIII) cDNA; the 3′ untranslated sequence from the hGH gene; sequences for plasmid replication in E. coli; and the amp gene for ampicillin selection in E. coli. FIG. 5B shows in vivo expression of human Factor VIII in nude mice implanted with human fibroblasts transfected with pXF8.941. Each point is the average (± standard error) of hFVIII ELISA values determined for plasma samples taken from individual mice. Open circles are nude mice implanted with a hFVIII expressing human fibroblast clone transfected with pXF8.941. Closed circles are control animals injected with saline.

FIG. 6 shows in vivo expression of human Factor VIII in nude mice implanted with human fibroblasts transfected with pXF8.971, which has the regulatory elements indicated. Each point is the average of hFVIII ELISA values determined for plasma samples taken from individual mice at the times indicated on the X-axis. Each line represents a different clone implanted into 5 nude mice.

FIG. 7 shows in vivo expression of human Factor VIII in nude mice implanted with human fibroblasts transfected with pXF8.914, which has the regulatory elements indicated. Each point is the average of hFVIII ELISA values determined for plasma samples taken from individual mice at the times indicated on the X-axis. Each line represents a different clone implanted into 5 nude mice.

FIG. 8 shows in vivo expression of human Factor VIII in nude mice implanted with human fibroblasts transfected with pXF8.973, which has the regulatory elements indicated. Each point is the average of hFVIII ELISA values determined for plasma samples taken from individual mice at the times indicated on the X-axis. Each line represents a different clone implanted into 5 nude mice.

FIG. 9 shows in vivo expression of human Factor VIII in nude mice implanted with human fibroblasts transfected with pXF8.751, which has the regulatory elements indicated. Each point is the average of hFVIII ELISA values determined for plasma samples taken from individual mice at the times indicated on the X-axis. Each line represents a different clone implanted into 5 nude mice.

FIG. 10 shows in vivo expression of human Factor VIII in nude mice implanted with human fibroblasts transfected with pXF8.753, which has the regulatory elements indicated. Each point is the average of hFVIII ELISA values determined for plasma samples taken from individual mice at the times indicated on the X-axis. Each line represents a different clone implanted into 5 nude mice.

FIG. 11 shows in vivo expression of human Factor VIII in nude mice implanted with human fibroblasts transfected with pXF8.1111, which has the regulatory elements indicated. Each point is the average of hFVIII ELISA values determined for plasma samples taken from individual mice at the times indicated on the X-axis. Each line represents a different clone implanted into 5 nude mice.

FIG. 12 shows in vivo expression of human Factor VIII in nude mice implanted with human fibroblasts transfected with pXF8.831, which has the regulatory elements indicated. Each point is the average of hFVIII ELISA values determined for plasma samples taken from individual mice. Each line represents a different clone implanted into 5 nude mice.

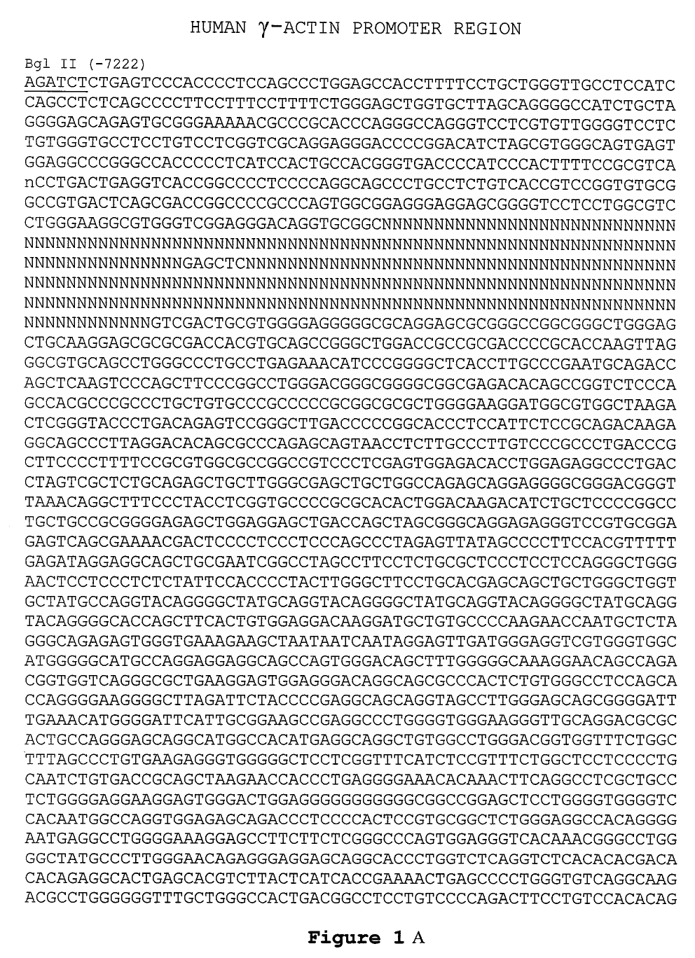

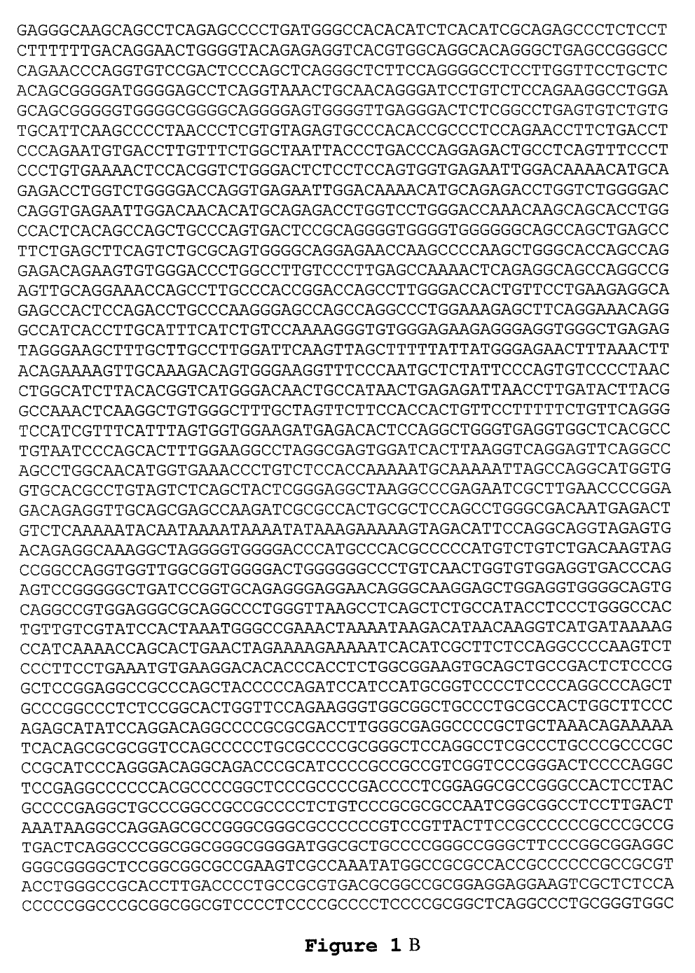

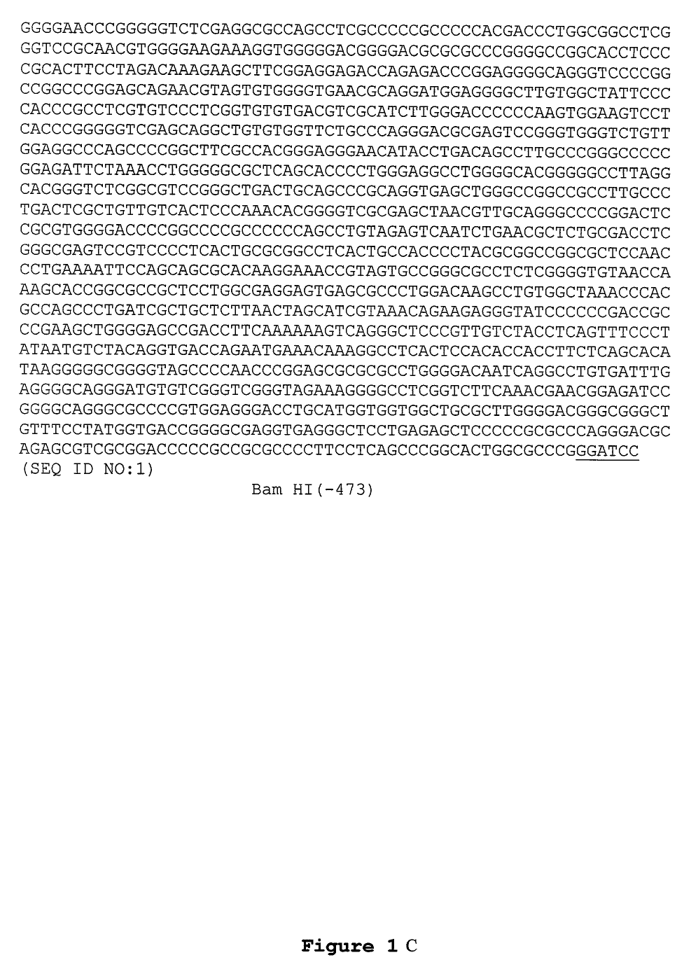

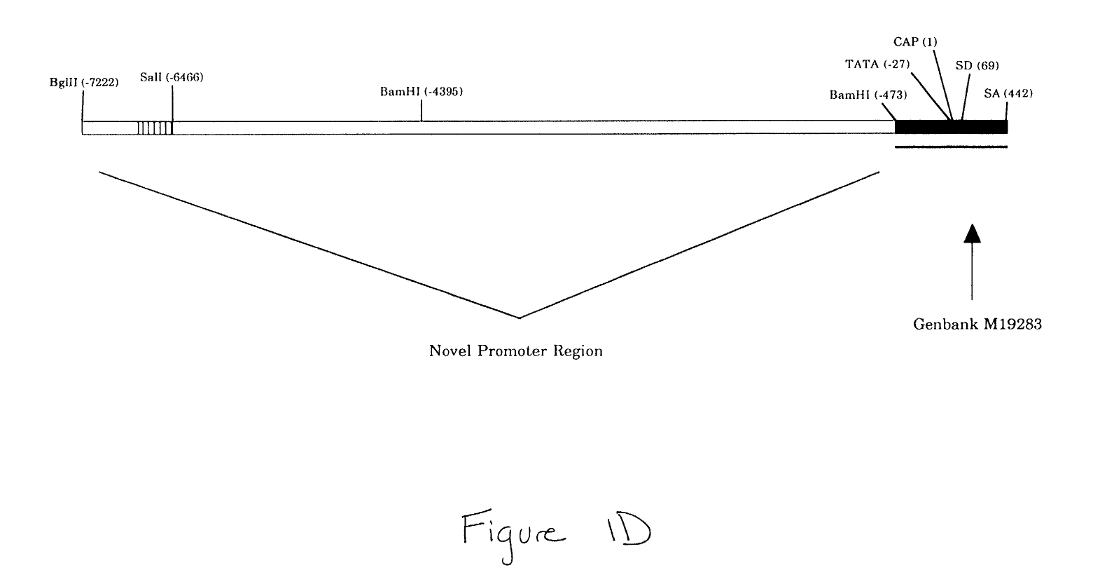

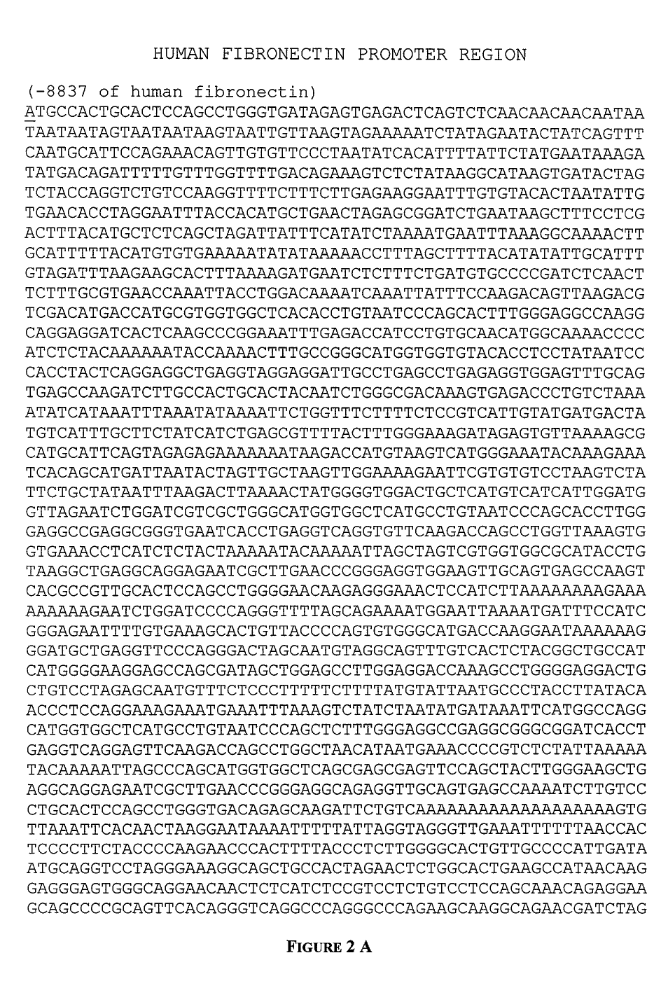

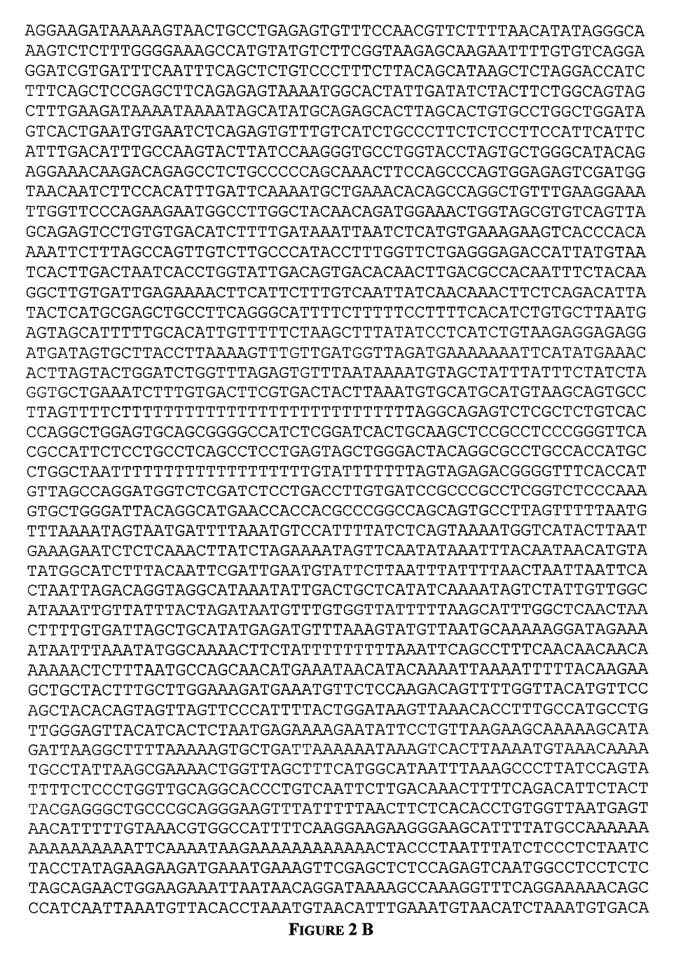

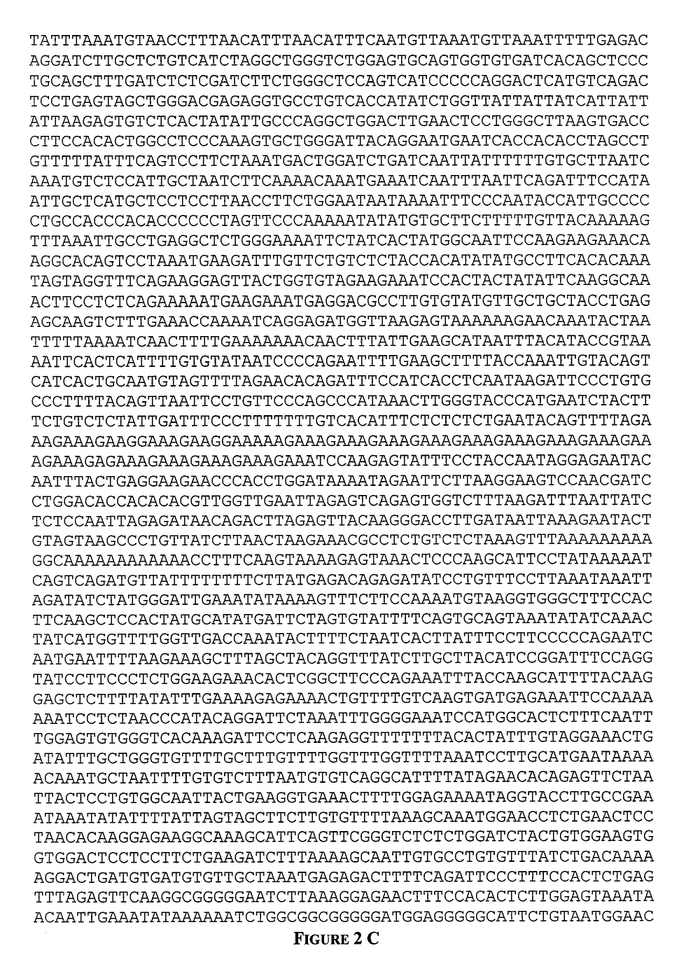

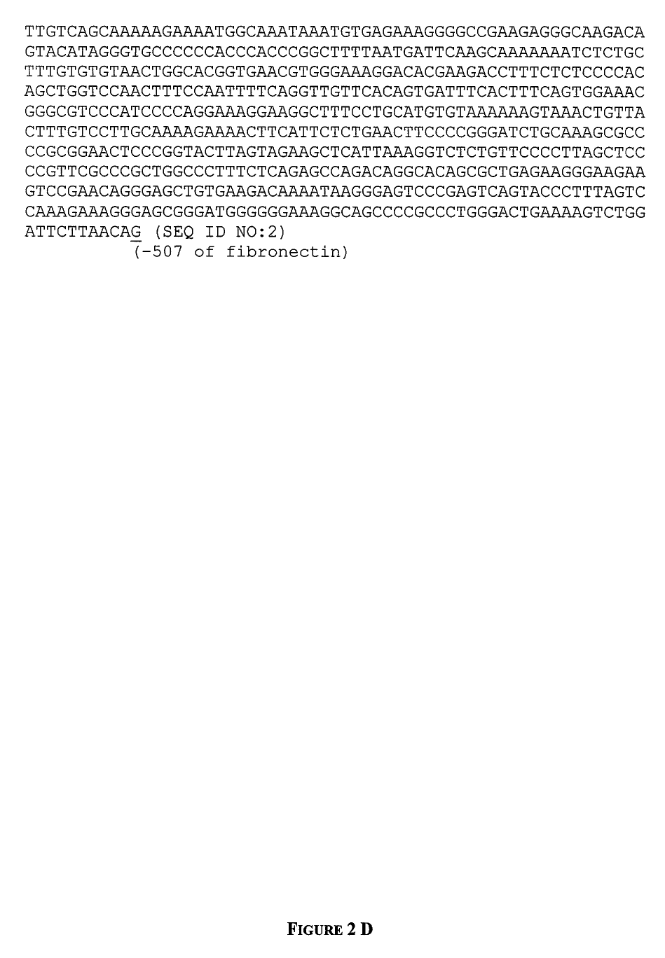

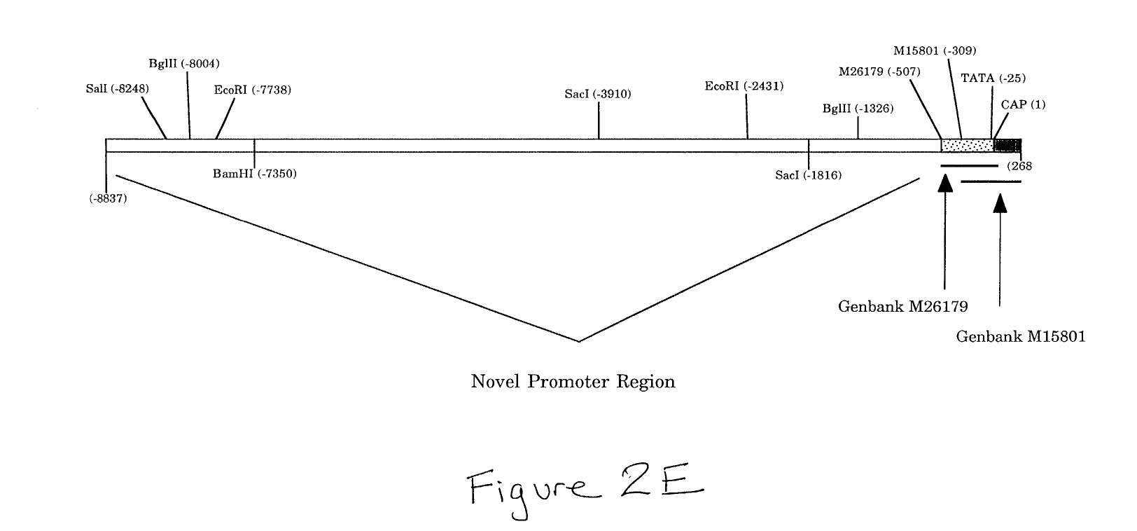

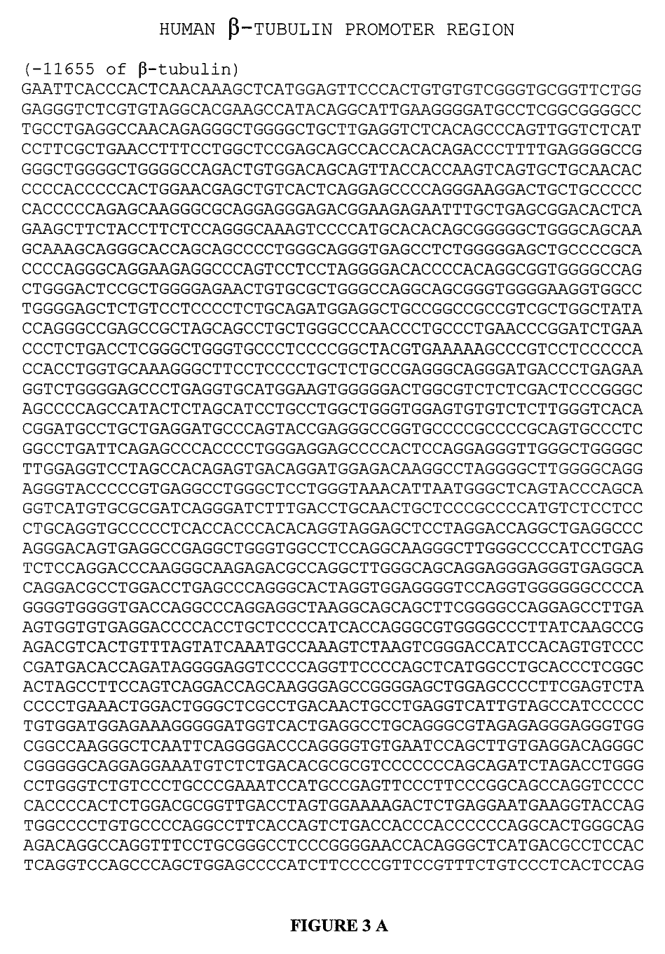

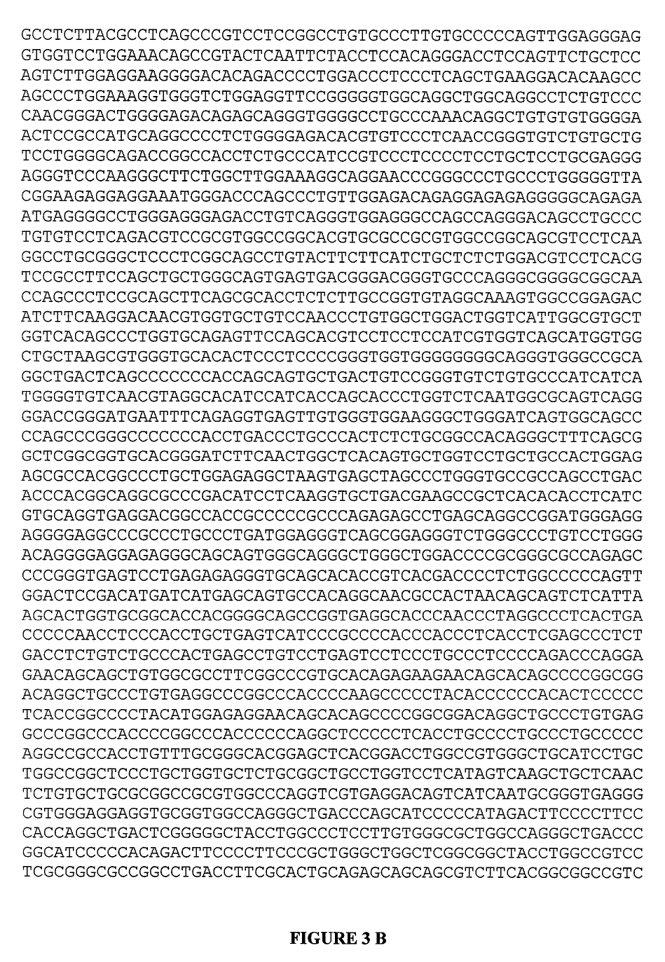

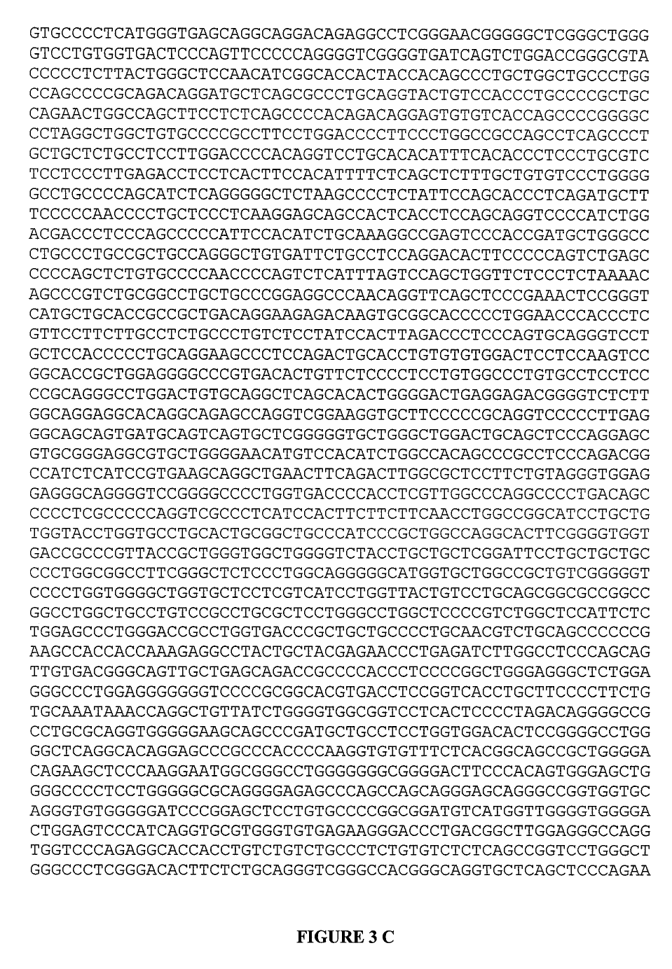

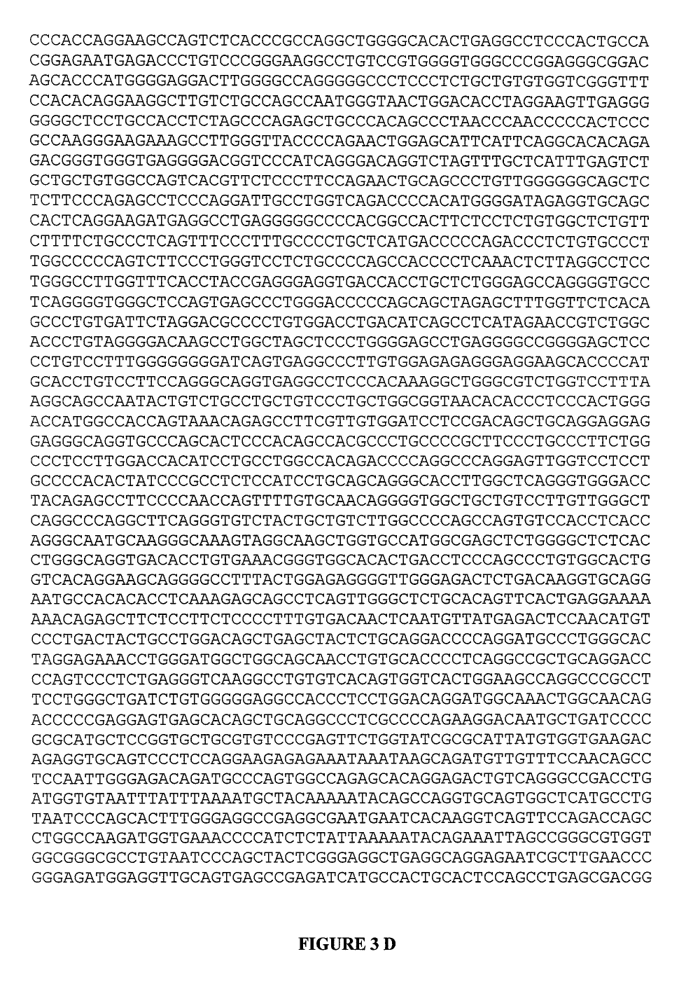

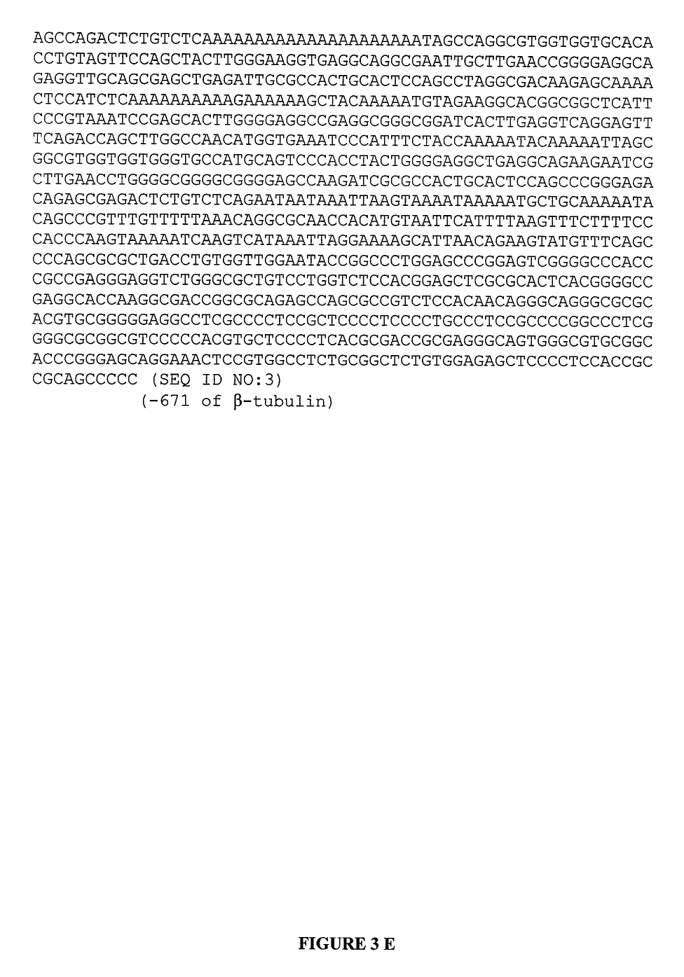

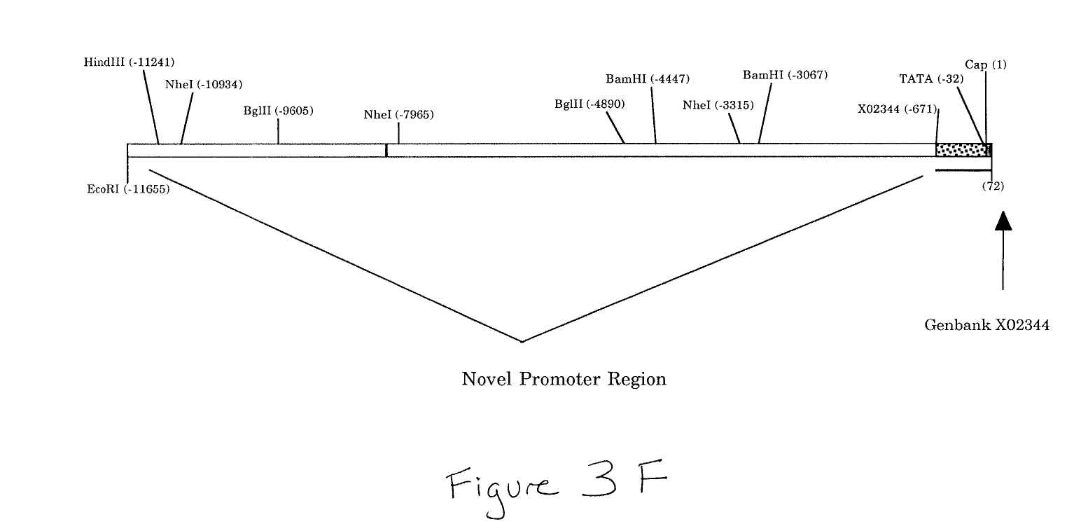

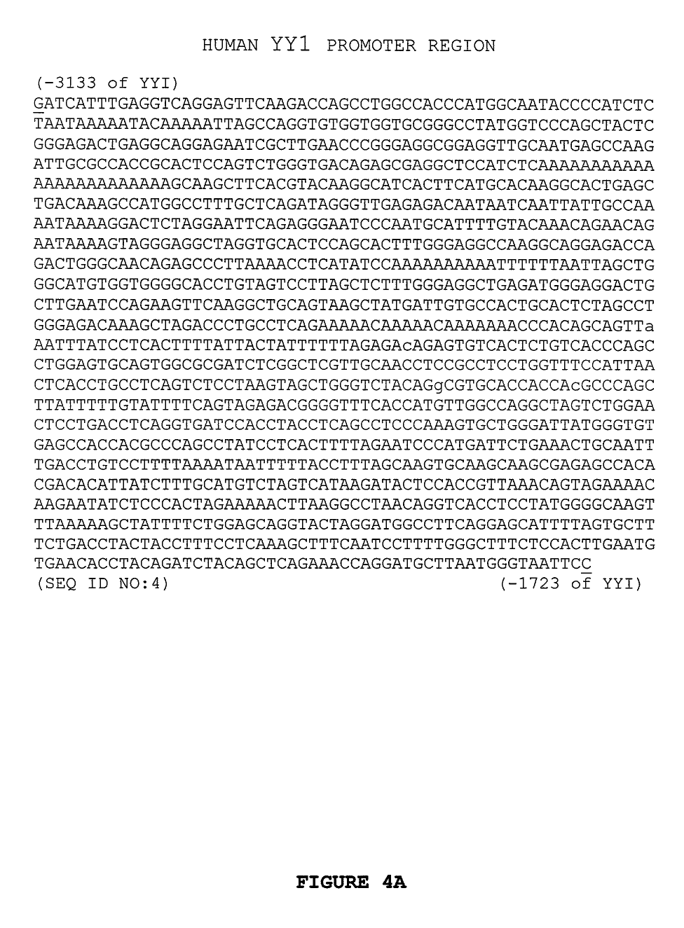

### DETAILED DESCRIPTION

Constructs, e.g., plasmid constructs, have been developed for the expression, particularly long term expression, of a nucleic acid sequence, e.g., an exogenous nucleic acid sequence, in a cell, e.g., a human cell, preferably a human fibroblast. The constructs can be used to transfect mammalian cells, e.g., human cells, preferably fibroblasts, e.g., primary fibroblasts, either in vitro or in vivo, to thereby produce a product (e.g., a polypeptide) encoded by the exogenous nucleic acid sequence and/or provide the product to a subject, e.g., a human. For example, stably transfected clonal skin fibroblasts expressing the human clotting factor FVIII (hFVIII) led to long-term (over 1 year) delivery of the clotting factor to the systemic circulation of mice. Plasma levels of 100-300 mU/mL were stably maintained, corresponding to 10-30% of the normal human level. Administration of stably transfected clonal skin fibroblasts expressing hFVIII can be used successfully for the treatment of, e.g., hemophilia A.

DNA Constructs

DNA constructs described herein, which include a promoter region and a heterologous nucleic acid (and preferably, additional regulatory elements), are constructed using standard genetic engineering techniques that are known in the art, e.g., restriction enzyme digestion, nucleic acid ligation, and polymerase chain reaction (PCR). Such routine techniques are described, e.g., in standard laboratory molecular biology treatises, e.g., Sambrook et al. Molecular Cloning: A Laboratory Manual, 3d ed., 2001, Cold Spring Harbor, which is hereby incorporated in its entirety.

Such constructs can be used to transfect primary or secondary cells in which a protein, e.g., an encoded protein, is to be produced. Alternatively, infectious vectors, such as retroviral, herpes, lentivirus, adenovirus, adenovirus-associated, mumps and poliovirus vectors, can be used for this purpose.

A construct described herein can include one or more additional sequences necessary for expression of the heterologous nucleic acid sequence. For example, a construct described herein can include a 3′ untranslated sequence (UTS), e.g., a polyadenylation site. A preferred 3′ UTS is the human growth hormone 3′ UTS.

A construct described herein can also include a nucleic acid sequence encoding a selectable marker used to confer a selectable phenotype upon introduction into bacteria, and/or into mammalian cells, e.g., primary or secondary cells. A variety of selectable markers can be incorporated into primary or secondary cells. For example, a selectable marker which confers a selectable phenotype such as drug resistance, nutritional auxotrophy, resistance to a cytotoxic agent or expression of a surface protein, can be used. Selectable marker genes which can be used include neo, gpt, dhfr, ada, pac (puromycin), hyg and hisD. The selectable phenotype conferred can make it possible to identify and isolate recipient primary or secondary cells. A selectable marker can be carried on a construct described herein or on a separate construct which can be co-transfected with a construct described herein into a cell or a subject.

Promoter Regions and Other Regulatory Elements

The promoter regions described herein can include known promoter sequence and/or novel promoter region sequence described herein, e.g., at least two contiguous nucleotides from the sequence of SEQ ID NO: 1, SEQ ID NO:2, SEQ ID NO:3, or SEQ ID NO:4. Preferably, a promoter region described herein includes at least 500, 1000, 1500, 2000, 3000, 4000, 5000, 6000, or more, nucleotides from the sequence shown as SEQ ID NO: 1, SEQ ID NO:2, SEQ ID NO:3, or SEQ ID NO:4.

In preferred embodiments, a promoter region described herein includes at least two contiguous nucleotides, but less than the full length sequence, of the sequence shown in SEQ ID NO: 1, SEQ ID NO:2, SEQ ID NO:3, or SEQ ID NO:4.

In preferred embodiments, a construct described herein includes at least one of the following elements:

Enhancers

The inventors have found that long term expression of a nucleic acid sequence encoding a desired gene product can be enhanced by the placement, preferably the specific placement, of an enhancer element in the constructs described herein. For example, expression of a desired gene product is enhanced by placement of an enhancer, e.g., a CMV enhancer, in a construct described herein. Preferably, the enhancer is placed, e.g., between cap −300 base pairs to cap −1100 base pairs, preferably at cap −825 base pairs, in a construct described herein that includes a human gamma-actin promoter region; between about cap −100 base pairs to cap −1000 base pairs, preferably at about cap −860 base pairs, in a construct described herein that includes a human YY1 promoter region; between cap −50 base pairs to cap −700 base pairs, preferably at cap −415 to cap −515 base pairs, in a construct described herein that includes a human fibronectin promoter region; or between cap −50 base pairs to cap −500 base pairs in a construct described herein that includes a human beta-actin promoter region.

5′ UTS Elements

It has also been discovered that long term expression of a nucleic acid sequence encoding a desired gene product can be enhanced by the use of a 5′ UTS element in the constructs described herein. For example, expression of a desired gene product is enhanced by placement of a 5′ UTS fusion (preferably of a particular sequence length), or a heterologous 5′ UTS, e.g., an aldolase 5′ UTS, an EF1-alpha 5′ UTS, or a beta-actin 5′ UTS, in a construct described herein. A 5′ UTS sequence element of the constructs described herein is preferably between about 1 and 500 base pairs in length, more preferably between about 25 and 250 base pairs in length, even more preferably between 25 and 150 base pair in length. 5′ UTS sequences for the various genes described herein are known and can easily be found, e.g., by searching a database of known gene sequences for 5′ UTS sequence of a particular gene, e.g., by searching the online GenBank database of gene sequences, e.g., by using the gene name as a keyword.

In preferred embodiments, a construct described herein that includes a γ-actin promoter region also includes a γ-actin 5′ UTS fusion of cap +25 to cap +50 base pairs in length, and a heterologous 5′ UTS, e.g., an aldolase 5′ UTS, or an EF1-alpha 5′ UTS. In a particularly preferred embodiment, the construct includes a γ-actin 5′ UTS fusion of about cap +25 base pairs in length fused to an aldolase 5′ UTS.

In other preferred embodiments, a construct described herein that includes a β-tubulin promoter region also includes a heterologous 5′ UTS, e.g., an aldolase 5′ UTS, a β-actin 5′ UTS, or an EF1-alpha 5′ UTS. A preferred 5′ UTS for a β-tubulin-based construct described herein is an EF1-alpha 5′ UTS.

In another preferred embodiment, a construct described herein that includes a fibronectin promoter region also includes a 5′ UTS fusion of about cap +140 to cap +270 base pairs in length or a heterologous 5′ UTS, e.g., an aldolase 5′ UTS, a β-actin 5′ UTS, or an EF1-alpha 5′ UTS, preferably a beta-actin 5′ UTS fused to an EF1-alpha U1 intron.

Preferably, a construct described herein that includes a β-actin promoter region also includes a β-actin 5′ UTS fusion, preferably of between about cap +20 to cap +120 base pairs in length, more preferably of about cap +50 to cap +100 base pairs, even more preferably of about cap +77 base pairs in length; and/or a heterologous 5′ UTS, e.g., an aldolase 5′ UTS. The sequence of a human β-actin promoter region can be found, e.g., at Genbank Accession No. Y00474.

Intron

It has also been discovered that long term expression of a nucleic acid sequence encoding a desired gene product can be enhanced by the placement of a heterologous U1 intron in a construct described herein. U1 introns are described, e.g., in Korb & Johnson, 1993, Nucleic Acids Res. 21(25):5901-8. Preferred U1 introns include a EF1-α U1 intron, GAPDH U1 intron and a β-actin U1 intron. In some preferred embodiments, a construct described herein includes a heterologous U1 intron in combination, e.g., fused to, a 5′ UTS region. For example, a preferred construct described herein that includes a fibronectin promoter region preferably also includes an EF1-α U1 intron fused to a β-actin 5′ UTS; and a preferred construct described herein that includes a β-actin promoter region preferably also includes an EF1-α U1 intron and a β-actin 5′ UTS fusion, preferably of about cap +50 to cap +100 base pairs in length.

MAR

The addition of MAR's has been found to enhance long term expression in constructs described herein. A preferred MAR is a βI MAR. In preferred embodiments, a construct described herein that includes a γ-actin, a YY1, a fibronectin, or a β-actin promoter region also includes at least one MAR, preferably 5′ and 3′ βI MAR's.

Heterologous Nucleic Acids

The constructs described herein can include a promoter region described herein, operably linked to a heterologous nucleic acid. The heterologous nucleic acids of the constructs described herein can be heterologous nucleic acids which can affect the expression of a protein, or a portion thereof, useful to treat an existing condition or prevent it from occurring. For example, the heterologous nucleic acid can encode a protein, or a portion thereof, useful to treat an existing condition or prevent it from occurring. The heterologous nucleic acid can also be a sequence, e.g., a regulatory sequence, e.g., a promoter or enhancer, or a sequence encoding a transcription factor, that increases or decreases the expression of a protein, or a portion thereof, useful to treat an existing condition or prevent it from occurring.

A heterologous nucleic acid of the constructs described herein can be an entire gene encoding an entire desired protein or a gene portion which encodes, for example, the active or functional portion(s) of the protein. The protein can be, for example, a hormone (e.g., human growth hormone), a cytokine, an antigen, an antibody, an enzyme, a clotting factor, e.g., Factor VIII or Factor XI, a transport protein, a receptor, a regulatory protein, a structural protein, or a protein which does not occur in nature. The heterologous nucleic acids of the constructs described herein can encode one or more therapeutic proteins. Preferably, the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, β-galactosidase, N-acetylgalactosamine-4-sulfatase (arylsulfatase B), β-glucuronidase or a biologically active fragment thereof.

A heterologous nucleic acid can also include a nucleic acid sequence optimized for expression in mammalian cells, e.g., human cells.

After introduction into primary or secondary cells, a construct described herein, or a portion thereof, can be stably incorporated into the recipient cell's genome, from which the heterologous nucleic acid is expressed or otherwise functions. The construct may also exist episomally within the primary or secondary cells.

Transfected or Infected Cells

Primary and secondary cells to be transfected with the nucleic acids and/or constructs described herein can be obtained from a variety of tissues and can include cell types which can be maintained and propagated in culture. For example, primary and secondary cells which can be transfected or infected include fibroblasts, keratinocytes, epithelial cells (e.g., mammary epithelial cells, intestinal epithelial cells), endothelial cells, glial cells, neural cells, a cell comprising a formed element of the blood (e.g., lymphocytes, bone marrow cells), muscle cells and precursors of these somatic cell types. Primary cells are preferably obtained from the individual to whom the transfected or infected primary or secondary cells are administered. However, primary cells may be obtained from a donor (other than the recipient) of the same species or another species (e.g., mouse, rat, rabbit, cat, dog, pig, cow, bird, sheep, goat, horse).

Primary or secondary cells of vertebrate, particularly mammalian, origin can be transfected or infected with nucleic acids and/or constructs described herein encoding a therapeutic protein and produce an encoded therapeutic protein stably and reproducibly, both in vitro and in vivo, over extended periods of time. In addition, the transfected or infected primary and secondary cells can express the encoded product in vivo at physiologically relevant levels, cells can be recovered after implantation and, upon reculturing, to grow and display their preimplantation properties.

The transfected or infected primary or secondary cells may also include DNA encoding a selectable marker which confers a selectable phenotype upon them, facilitating their identification and isolation. Methods for producing transfected primary, secondary cells which stably express exogenous DNA, clonal cell strains and heterogenous cell strains of such transfected cells, methods of producing the clonal and heterogenous cell strains, and methods of treating or preventing an abnormal or undesirable condition through the use of populations of transfected primary or secondary cells are part of the present invention. Primary and secondary cells which can be transfected or infected include fibroblasts, keratinocytes, epithelial cells (e.g., mammary epithelial cells, intestinal epithelial cells), endothelial cells, glial cells, neural cells, a cell comprising a formed element of the blood (e.g., a lymphocyte, a bone marrow cell), muscle cells and precursors of these somatic cell types. Primary cells are preferably obtained from the individual to whom the transfected or infected primary or secondary cells are administered. However, primary cells may be obtained from a donor (other than the recipient) of the same species or another species (e.g., mouse, rat, rabbit, cat, dog, pig, cow, bird, sheep, goat, horse). Transformed or immortalized cells can also be used e.g., a Bowes Melanoma cell (ATCC Accession No. CRL 9607), a Daudi cell (ATCC Accession No. CCL 213), a HeLa cell and a derivative of a HeLa cell (ATCC Accession Nos. CCL 2, CCL2.1, and CCL 2.2), a HL-60 cell (ATCC Accession No. CCL 240), a HT-1080 cell (ATCC Accession No. CCL 121), a Jurkat cell (ATCC Accession No. TIB 152), a KB carcinoma cell (ATCC Accession No. CCL 17), a K-562 leukemia cell (ATCC Accession No. CCL 243), a MCF-7 breast cancer cell (ATCC Accession No. BTH 22), a MOLT-4 cell (ATCC Accession No. 1582), a Namalwa cell (ATCC Accession No. CRL 1432), a Raji cell (ATCC Accession No. CCL 86), a RPMI 8226 cell (ATCC Accession No. CCL 155), a U-937 cell (ATCC Accession No. CRL 1593), WI-38VA13 sub line 2R4 cells (ATCC Accession No. CLL 75.1), a CCRF-CEM cell (ATCC Accession No. CCL 119) and a 2780AD ovarian carcinoma cell (Van Der Blick et al., Cancer Res. 48: 5927-5932, 1988), as well as heterohybridoma cells produced by fusion of human cells and cells of another species. In another embodiment, the immortalized cell line can be a cell line other than a human cell line, e.g., a CHO cell line or a COS cell line. In a preferred embodiment, the cell is a non-transformed cell. In various preferred embodiments, the cell is a mammalian cell, e.g., a primary or secondary mammalian cell, e.g., a fibroblast, a hematopoietic stem cell, a myoblast, a keratinocyte, an epithelial cell, an endothelial cell, a glial cell, a neural cell, a cell comprising a formed element of the blood, a muscle cell and precursors of these somatic cells. In a most preferred embodiment, the cell is a secondary human fibroblast.

Alternatively, the nucleic acids and/or constructs described herein can be delivered into any of the cell types discussed above by a viral vector infection. Viruses known to be useful for gene transfer include adenoviruses, adeno-associated virus, herpes virus, mumps virus, poliovirus, retroviruses, Sindbis virus, and vaccinia virus such as canary pox virus. Use of viral vectors is well known in the art: see e.g., Robbins and Ghizzani, Mol. Med. Today 1:410-417, 1995. A cell which has an exogenous DNA introduced into it by a viral vector is referred to as an “infected cell.”

The invention also includes the genetic manipulation of a cell to produce a therapeutic protein. A cell is transfected with a nucleic acid sequence, e.g., a construct described herein, that causes or alters the production of a gene product, or a portion thereof. The product can be useful to treat an existing condition, prevent it from occurring, or delaying its onset.

In some preferred embodiments, the construct transfected into subject cells, e.g., human fibroblasts, can include an entire gene; a coding sequence of a gene, encoding an entire desired protein; or a portion thereof which encodes, for example, the active or functional portion(s) of the protein. The protein can be, for example, a hormone, a cytokine, an antigen, an antibody, an enzyme, a clotting factor, a transport protein, a receptor, a regulatory protein, a structural protein, or a protein which does not occur in nature. The construct may also encode a therapeutic RNA or an active or functional portion(s) thereof.

In other embodiments, the subject cells, e.g., human fibroblasts, can be genetically engineered to contain an exogenous DNA sequence which includes a regulatory sequence including one or more of: a promoter region, an enhancer, an intron, an untranslated sequence (UAS), an MAR or a transcription binding site, e.g., the cells can be transfected with a construct described herein. The construct is targeted to result in the insertion of the regulatory sequence of the construct, placing a targeted endogenous gene under its control (for example, by insertion of either a promoter or an enhancer, or both, upstream of the endogenous gene or regulatory region). Optionally, the targeting event can simultaneously result in the deletion of an endogenous regulatory sequence, such as the deletion of a tissue-specific negative regulatory sequence, of a gene. The targeting event can replace an existing regulatory sequence; for example, a tissue-specific enhancer can be replaced by an enhancer (e.g., an enhancer contained in a construct described herein) that has broader or different cell-type specificity than the naturally-occurring elements, or displays a pattern of regulation or induction that is different from the corresponding nontransfected or noninfected cell. In this regard, the naturally occurring sequences are deleted and new sequences are added. In some cases, the endogenous regulatory sequences are not removed or replaced but are disrupted or disabled by the targeting event, such as by targeting the exogenous sequences within the endogenous regulatory elements. Introduction of a regulatory sequence by homologous recombination can result in a cell expressing a therapeutic protein which it does not normally express. In addition, targeted introduction of a regulatory sequence can be used for cells which make or contain the therapeutic protein but in lower quantities than normal (in quantities less than the physiologically normal lower level) or in defective form, and for cells which make the therapeutic protein at physiologically normal levels, but are to be augmented or enhanced in their content or production. Examples of methods of activating an endogenous coding sequence are described in U.S. Pat. No. 5,641,670, U.S. Pat. No. 5,733,761, U.S. Pat. No. 5,968,502, U.S. Pat. No. 6,214,622, U.S. Pat. No. 6,270,989, and U.S. Pat. No. 6,242,218, the contents of which are incorporated herein by reference.

Transfection of Primary or Secondary Cells and Production of Clonal or Heterogenous Cell Strains

Vertebrate tissue can be obtained by standard methods such as punch biopsy or other surgical methods of obtaining a tissue source of the primary cell type of interest. For example, punch biopsy is used to obtain skin as a source of fibroblasts or keratinocytes. A mixture of primary cells is obtained from the tissue, using known methods, such as enzymatic digestion. If enzymatic digestion is used, enzymes such as collagenase, hyaluronidase, dispase, pronase, trypsin, elastase and chymotrypsin can be used.

The resulting primary cell mixture can be transfected directly or it can be cultured first, removed from the culture plate and resuspended before transfection is carried out. Primary cells or secondary cells are combined with exogenous DNA such as the constructs described herein to be stably integrated into their genomes and, optionally, DNA encoding a selectable marker, and treated in order to accomplish transfection. The exogenous DNA and selectable marker-encoding DNA are each on a separate construct or on a single construct and an appropriate quantity of DNA to ensure that at least one stably transfected cell containing and appropriately expressing exogenous DNA is produced. In general, 0.1 to 500 μg DNA is used.

Primary or secondary cells can be transfected by electroporation. Electroporation is carried out at appropriate voltage and capacitance (and time constant) to result in entry of the DNA construct(s) into the primary or secondary cells. Electroporation can be carried out over a wide range of voltages (e.g., 50 to 2000 volts) and capacitance values (e.g., 60-300 μFarads). Total DNA of approximately 0.1 to 500 μg is generally used.

Primary or secondary cells can be transfected using microinjection. Alternatively, known methods such as calcium phosphate precipitation, modified calcium phosphate precipitation and polybrene precipitation, liposome fusion and receptor-mediated gene delivery can be used to transfect cells. A stably, transfected cell is isolated and cultured and subcultivated, under culturing conditions and for sufficient time, to propagate the stably transfected secondary cells and produce a clonal cell strain of transfected secondary cells. Alternatively, more than one transfected cell is cultured and subcultured, resulting in production of a heterogenous cell strain.

Transfected primary or secondary cells undergo a sufficient number of doublings to produce either a clonal cell strain or a heterogenous cell strain of sufficient size to provide the therapeutic protein to an individual in effective amounts. In general, for example, 0.1 cm² of skin is biopsied and assumed to contain 100,000 cells; one cell is used to produce a clonal cell strain and undergoes approximately 27 doublings to produce 100 million transfected secondary cells. If a heterogenous cell strain is to be produced from an original transfected population of approximately 100,000 cells, only 10 doublings are needed to produce 100 million transfected cells.

The number of required cells in a transfected clonal or heterogenous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient. To put these factors in perspective, to deliver therapeutic levels of human growth hormone in an otherwise healthy 10 kg patient with isolated growth hormone deficiency, approximately one to five hundred million transfected fibroblasts would be necessary (the volume of these cells is about that of the very tip of the patient's thumb).

Episomal Expression

DNA sequences that are present within the cell yet do not integrate into the genome are referred to as episomes. Recombinant episomes may be useful in at least three settings: 1) if a given cell type is incapable of stably integrating the exogenous DNA; 2) if a given cell type is adversely affected by the integration of DNA; and 3) if a given cell type is capable of improved therapeutic function with an episomal rather than integrated DNA.

Using transfection and culturing as described herein, exogenous DNA (such as the constructs described herein) in the form of episomes can be introduced into vertebrate primary and secondary cells. Plasmids can be converted into such an episome by the addition DNA sequences for the Epstein-Barr virus origin of replication and nuclear antigen (Yates, J. L. Nature 319:780-7883 (1985)). Alternatively, vertebrate autonomously replicating sequences can be introduced into the construct (Weidle, U. H. Gene 73(2):427-437 (1988). These and other episomally derived sequences can also be included in DNA constructs without selectable markers, such as pXGH5 (Selden et al., Mol Cell Biol. 6:3173-3179, 1986). The episomal exogenous DNA is then introduced into primary or secondary vertebrate cells as described in this application (if a selective marker is included in the episome a selective agent is used to treat the transfected cells).

Implantation of Clonal Cell Strain or Heterogenous Cell Strain of Transfected Secondary Cells

The transfected or infected cells produced as described above can be introduced into an individual to whom the therapeutic protein is to be delivered, using known methods. The clonal cell strain or heterogenous cell strain is then introduced into an individual, using known methods, using various routes of administration and at various sites (e.g., renal subcapsular, subcutaneous, central nervous system (including intrathecal), intravascular, intrahepatic, intrasplanchnic, intraperitoneal (including intraomental, or intramuscular implantation). In a preferred embodiment, the clonal cell strain or heterogeneous cell strain is introduced into the omentum.

The omentum is a membranous structure containing a sheet of fat. Usually, the omentum is a fold of peritoneum extending from the stomach to adjacent abdominal organs. The greater omentum is attached to the inferior edge of the stomach and hangs down in front of the intestines. The other edge is attached to the transverse colon. The lesser omentum is attached to the superior edge of the stomach and extends to the undersurface of the liver. The cells may be introduced into any part of the omentum by surgical implantation, laparoscopy or direct injection, e.g., via CT-guided needle or ultrasound. Once implanted in the individual, the cells produce the therapeutic product encoded by the exogenous DNA or are affected by the exogenous DNA itself. For example, an individual who has been diagnosed with Hemophilia A, a bleeding disorder that is caused by a deficiency in Factor VIII, a protein normally found in the blood, is a candidate for a gene therapy treatment. In another example, an individual who has been diagnosed with Hemophilia B, a bleeding disorder that is caused by a deficiency in Factor IX, a protein normally found in the blood, is a candidate for a gene therapy treatment. The patient has a small skin biopsy performed. This is a simple procedure which can be performed on an out-patient basis. The piece of skin, approximately the size of a match head, is taken, for example, from under the arm and requires about one minute to remove. The sample is processed, resulting in isolation of the patient's cells and genetically engineered to produce a missing or underexpressed protein or peptide, e.g., the missing Factor IX or Factor VIII. Based on the age, weight, and clinical condition of the patient, the required number of cells are grown in large-scale culture. The entire process requires 4-6 weeks and, at the end of that time, the appropriate number, e.g., approximately 100-500 million genetically engineered cells are introduced into the individual, once again as an outpatient (e.g., by injecting them back under the patient's skin). The patient is now capable of producing his or her own Factor IX or Factor VIII and is no longer a hemophiliac.

A similar approach can be used to treat other conditions or diseases. For example, short stature can be treated by administering human growth hormone to an individual by implanting primary or secondary cells which express human growth hormone; anemia can be treated by administering erythropoietin (EPO) to an individual by implanting primary or secondary cells which express EPO; or diabetes can be treated by administering glucagon-like peptide-1 (GLP-1) to an individual by implanting primary or secondary cells which express GLP-1. A lysosomal storage disease (LSD) can be treated by this approach. LSD's represent a group of at least 41 distinct genetic diseases, each one representing a deficiency of a particular protein that is involved in lysosomal biogenesis. A particular LSD can be treated by administering a lysosomal enzyme to an individual by implanting primary or secondary cells which express the lysosomal enzyme, e.g., Fabry Disease can be treated by administering α-galactosidase to an individual by implanting primary or secondary cells which express α-galactosidase; Gaucher disease can be treated by administering glucocerebrosidase to an individual by implanting primary or secondary cells which express β-glucocerebrosidase; MPS (mucopolysaccharidosis) type I (Hurler-Scheie syndrome) can be treated by administering α-L-iduronidase to an individual by implanting primary or secondary cells which express α-L-iduronidase; MPS type II (Hunter syndrome) can be treated by administering iduronate-2-sulfatase to an individual by implanting primary or secondary cells which express iduronate-2-sulfatase; MPS type III-A (Sanfilipo A syndrome) can be treated by administering Heparan N-sulfatase to an individual by implanting primary or secondary cells which express Heparan N-sulfatase; MPS type III-B (Sanfilipo B syndrome) can be treated by administering α-N-acetylglucosaminidase to an individual by implanting primary or secondary cells which express α-N-acetylglucosaminidase; MPS type III-C (Sanfilipo C syndrome) can be treated by administering acetyl coenzyme A:α-glucosaminide acetyltransferase to an individual by implanting primary or secondary cells which express acetyl coenzyme A:α-glucosaminide acetyltransferase; MPS type III-D (Sanfilippo D syndrome) can be treated by administering N-acetylglucosamine-6-sulfatase to an individual by implanting primary or secondary cells which express N-acetylglucosamine-6-sulfatase; MPS type IV-A (Morquio A syndrome) can be treated by administering galactose-6-sulfatase to an individual by implanting primary or secondary cells which express galactose-6-sulfatase; MPS type IV-B (Morquio B syndrome) can be treated by administering β-galactosidase to an individual by implanting primary or secondary cells which express β-galactosidase; MPS type VI (Maroteaux-Lamy syndrome) can be treated by administering N-acetylgalactosamine-4-sulfatase (Arylsulfatase B) to an individual by implanting primary or secondary cells which express N-acetylgalactosamine4-sulfatase (Arylsulfatase B); MPS type VII (Sly syndrome) can be treated by administering β-glucuronidase to an individual by implanting primary or secondary cells which express β-glucuronidase.

The cells used for implantation will generally be patient-specific genetically engineered cells. It is possible, however, to obtain cells from another individual of the same species or from a different species. Use of such cells might require administration of an immunosuppressant, alteration of histocompatibility antigens, or use of a barrier device to prevent rejection of the implanted cells. For many diseases, this will be a one-time treatment and, for others, multiple gene therapy treatments will be required.

In one embodiment, cell therapy as described herein is based upon transfection of a patient's cells with an expression plasmid (encoding a therapeutic protein) and isolation of clonal populations of these cells producing the therapeutic protein at high levels. A clone producing suitable levels of protein is then isolated, expanded in culture, and implanted back into the original donor. Human dermal fibroblasts were chosen for the present studies because they are readily isolated from a simple skin biopsy, can be transfected efficiently using non-viral transfection methods, express high levels of appropriately processed therapeutic proteins, and can be expanded to large numbers in culture without spontaneous transformation.

Uses of Transfected or Infected Primary and Secondary Cells and Cell Strains

Transfected or infected primary or secondary cells or cell strains have wide applicability as a vehicle or delivery system for therapeutic proteins, such as enzymes, hormones, cytokines, antigens, antibodies, clotting factors, anti-sense RNA, regulatory proteins, transcription proteins, receptors, structural proteins, novel proteins and nucleic acid products, and engineered DNA. For example, transfected primary or secondary cells can be used to supply a therapeutic protein, including, but not limited to, Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, β-galactosidase, N-acetylgalactosamine-4-sulfatase (arylsulfatase B), β-glucuronidase, antitrypsin, calcitonin, glucocerebrosidase, low density lipoprotein (LDL), receptor IL-2 receptor and its antagonists, insulin, globin, immunoglobulins, catalytic antibodies, the interleukins, insulin-like growth factors, superoxide dismutase, immune responder modifiers, parathyroid hormone and interferon, nerve growth factors, tissue plasminogen activators, and colony stimulating factors. Alternatively, transfected primary and secondary cells can be used to immunize an individual (i.e., as a vaccine).

The wide variety of uses of constructs of the present invention can perhaps most conveniently be summarized as shown below. The constructs can be used to produce and/or deliver the following therapeutic products.

1. a secreted protein with predominantly systemic effects;

2. a secreted protein with predominantly local effects;

3. a membrane protein imparting new or enhanced cellular responsiveness;

4. membrane protein facilitating removal of a toxic product;

5. a membrane protein marking or targeting a cell;

6. an intracellular protein;

7. an intracellular protein directly affecting gene expression; and

8. an intracellular protein with autocrine effects.

Transfected or infected primary or secondary cells can be used to administer therapeutic proteins (e.g., hormones, enzymes, clotting factors) which are presently administered intravenously, intramuscularly or subcutaneously, which requires patient cooperation and, often, medical staff participation. When transfected or infected primary or secondary cells are used, there is no need for extensive purification of the polypeptide before it is administered to an individual, as is generally necessary with an isolated polypeptide. In addition, transfected or infected primary or secondary cells of the present invention produce the therapeutic protein as it would normally be produced.

An advantage to the use of transfected or infected primary or secondary cells is that by controlling the number of cells introduced into an individual, one can control the amount of the protein delivered to the body. In addition, in some cases, it is possible to remove the transfected or infected cells if there is no longer a need for the product. A further advantage of treatment by use of transfected or infected primary or secondary cells is that production of the therapeutic product can be regulated, such as through the administration of zinc, steroids or an agent which affects transcription of a protein, product or nucleic acid product or affects the stability of a nucleic acid product.

Transgenic Animals

A number of methods can be used to obtain transgenic, non-human mammals. A transgenic non-human mammal refers to a mammal that has gained an additional gene through the introduction of an exogenous nucleic acid sequence, i.e., transgene, into its own cells (e.g., both the somatic and germ cells), or into an ancestor's germ line.

There are a number of methods to introduce the exogenous DNA into the germ line (e.g., introduction into the germ or somatic cells) of a mammal. One method is by microinjection of a the gene construct into the pronucleus of an early stage embryo (e.g., before the four-cell stage) (Wagner et al., Proc. Natl. Acad. Sci. USA 78:5016 (1981); Brinster et al., Proc Natl Acad Sci USA 82:4438 (1985)). The detailed procedure to produce such transgenic mice has been described (see e.g., Hogan et al., Manipulating the Mouse Embryo, Cold Spring Harbour Laboratory, Cold Spring Harbour, NY (1986); U.S. Pat. No. 5,175,383 (1992)). This procedure has also been adapted for other mammalian species (e.g., Hammer et al., Nature 315:680 (1985); Murray et al., Reprod. Fert. Devl. 1:147 (1989); Pursel et al., Vet. Immunol. Histopath. 17:303 (1987); Rexroad et al., J. Reprod. Fert. 41(suppl):119 (1990); Rexroad et al., Molec. Reprod. Devl. 1:164 (1989); Simons et al., BioTechnology 6:179 (1988); Vize et al., J. Cell. Sci. 90:295 (1988); and Wagner, J. Cell. Biochem. 13B(suppl):164 (1989).

Another method for producing germ-line transgenic mammals is through the use of embryonic stem cells or somatic cells (e.g., embryonic, fetal or adult). The gene construct may be introduced into embryonic stem cells by homologous recombination (Thomas et al., Cell 51:503 (1987); Capecchi, Science 244:1288 (1989); Joyner et al., Nature 338: 153 (1989)). A suitable construct may also be introduced into the embryonic stem cells by DNA-mediated transfection, such as electroporation (Ausubel et al., Current Protocols in Molecular Biology, John Wiley & Sons (1987)). Detailed procedures for culturing embryonic stem cells (e.g. ESD3, ATCC# CCL-1934, ES-E14TG-2a, ATCC# CCL-1821, American Type Culture Collection, Rockville, Md.) and the methods of making transgenic mammals from embryonic stem cells can be found in Teratocarcinomas and Embryonic Stem Cells, A Practical Approach, ed. E. J. Robertson (IRL Press, 1987). Methods of making transgenic animals from somatic cells can be found, for example, in WO 97/07669, WO 97/07668 and U.S. Pat. No. 5,945,577.

In the above methods for the generation of a germ-line transgenic mammals, the construct may be introduced as a linear construct, as a circular plasmid, or as a vector which may be incorporated and inherited as a transgene integrated into the host genome. The transgene may also be constructed so as to permit it to be inherited as an extrachromosomal plasmid (Gassmann, M. et al., Proc. Natl. Acad. Sci. USA 92:1292 (1995)).

Nucleic Acid Fragments

A nucleic acid molecule of the invention can include only a portion of the nucleic acid sequence of SEQ ID NO: 1, 2, 3, or 4. A preferred fragment is one that has at least 10% of the activity of SEQ ID NO: 1, 2, 3, or 4, e.g., the activity to promote expression of an operably linked nucleic acid sequence or gene.

A nucleic acid fragment can include a sequence corresponding to a domain, region, or functional site described herein, e.g., a promoter element as described herein. A nucleic acid fragment also can include one or more domains, regions, or functional sites described herein.

In a preferred embodiment, the nucleic acid fragment is at least 50, 100, 500, 1000, 1500, 2000, 3000, 4000, 50000, or more nucleotides in length, and hybridizes under a stringent hybridization condition as described herein to a nucleic acid molecule of SEQ ID NO: 1, 2, 3, or 4. In a preferred embodiment, the nucleic acid fragment has at least 10%, preferably 20%, 30%, 40%, 50%, 60%, 70%, 80%, 90%, 95% or more of the activity, e.g., a promoter activity, of the sequence of SEQ ID NO: 1, 2, 3, or 4.

In a preferred embodiment, the nucleic acid fragment differs by at least one but less than 100, 50, 30, 20, 10, or nucleotides from the sequence of SEQ ID NO 1, 2, 3 or 4, and has at least 10%, preferably 20%, 30%, 40%, 50%, 60%, 70%, 80%, 90%, 95% or more of the activity, e.g., a promoter activity, of the sequence of SEQ ID NO: 1, 2, 3, or 4.

In another preferred embodiment, the fragment is a probe that is at least 5 or 10 and less than 500, 300, or 200 base pains in length, and more preferably is less than 100 or less than 50 base pairs in length. It should be identical, or differ by 1, or less than 5 or 10 bases, from a sequence disclosed herein. If alignment is needed for this comparison, the sequences should be aligned for maximum homology. “Looped” out sequences in the alignment from deletions, insertions, or mismatches, are considered differences.

In preferred embodiments, a nucleic acid includes a nucleotide sequence which is at least 300, 500, 1000, 2000, 3000, 4000, 5000, or more nucleotides in length and hybridizes under high stringency conditions described herein to a nucleic acid molecule of SEQ ID NO: 1, 2, 3, or 4.

An example of high stringency hybridization conditions is as follows: hybridization in 6×SSC at about 45° C., followed by one or more washes in 0.2×SSC, 0.1% SDS at 65° C.

Nucleic Acid Variants

The invention further encompasses nucleic acid molecules that differ from the nucleotide sequence shown in SEQ ID NO:1, 2, 3, or 4. Preferred variants have at least 10%, preferably 20%, 30%, 40%, 50%, 60%, 70%, 80%, 90%, 95% or more of the activity, e.g., a promoter activity, of the sequence of SEQ ID NO: 1, 2, 3, or 4.

In a preferred embodiment, the nucleic acid differs from that of SEQ ID NO: 1, 2, 3, or 4, e.g., as follows: by at least one but less than 10, 20, 30, or 40 nucleotides; at least one but less than 1%, 5%, 10% or 20% of the nucleotides in the subject nucleic acid. If necessary for this analysis, the sequences should be aligned for maximum homology. “Looped” out sequences from deletions, insertions, or mismatches, are considered differences.

Uses

The constructs, cells, and methods of the invention are useful for expressing a product, e.g., a polypeptide, normally expressed in a mammalian cell, or in cell culture (e.g. for commercial production of human proteins such as Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-625 sulfatase, galactose-6-sulfatase, β-galactosidase, N-acetylgalactosamine-4-sulfatase (arylsulfatase B), β-glucuronidase or a biologically active fragment thereof.

The constructs described herein are also useful for gene therapy. For example, a construct including a heterologous sequence encoding a selected protein can be introduced directly, e.g., via non-viral cell transfection or via a vector in to a cell, e.g., a transformed or a non-transformed cell, which can express the protein to create a cell which can be administered to a patient in need of the protein. Such cell-based gene therapy techniques are described in greater detail in co-pending US applications: U.S. Ser. No. 08/334,797; U.S. Ser. No. 08/231,439; U.S. Ser. No. 08/334,455; and U.S. Ser. No. 08/928,881, which are hereby expressly incorporated by reference in their entirety.

### EXAMPLES

### Example 1

### Construction and Characterization of Long-Term Expression Constructs

Basic expression plasmids were constructed using promoter region sequence from each of numerous known mammalian promoter sequences. Expression plasmids containing either a γ-actin, fibronectin, β-tubulin, YY1, or β-actin promoter region were identified as constructs to be further optimized with regard to: identification of novel promoter region sequence, as shown, e.g., in SEQ ID NO: 1-4; determination of optimal 5′ UTS length for a given construct (defined as cap+a defined range of bases), optimal heterologous 5′ UTS sequences, optimal U1 intron sequences, optimal enhancer sequence and placement thereof within a promoter region, and/or addition of MAR regions. All expression constructs were constructed using standard molecular biology techniques as described herein. Plasmids were constructed with an antibiotic resistance marker, e.g., an ampicillin resistance gene, to allow for selection in bacteria; and a eukaryotic drug resistance marker, e.g., a neomycin gene, to allow for selection in mammalian cells.

The constructs described herein were tested for long-term expression of human growth hormone (plasmids were built to express a hGH-N gene), or Factor VIII. The Factor VIII plasmids were built to express a Factor VIII cDNA optimized for expression in mammalian cells and containing a deletion of the Factor VIII B-domain, referred to herein as BDD Factor VIII, for example, as described in U.S. application Ser. No. 09/407,605, filed Sep. 28, 1999, pending. Human fibroblast strains were isolated, grown, and transfected under standard conditions. Mean expression levels were determined for each plasmid typically calculated from 12-25 clones per plasmid.

Based on expression data generated in vitro, clones were identified for further evaluation for long term expression in vivo. Clones were expanded until a suitable cell number was obtained for implantation into mice, for example, implantation into the omentum of nude mice. Quantitative in vivo expression levels, e.g., Factor VIII levels, were determined by analysis of blood samples drawn from experimental animals on a weekly basis. Constructs that resulted in long term expression, e.g., detectable expression for at least 4 weeks, preferably at least 6 weeks, more preferably at least 10, 20, 30, 40, 50 weeks or more, were identified, characterized, and are described herein. One specific, but non-limiting, example of such a construct is illustrated in Example 2. Other examples are shown in the accompanying drawings.

### Example 2

### Construction of pXF8.941

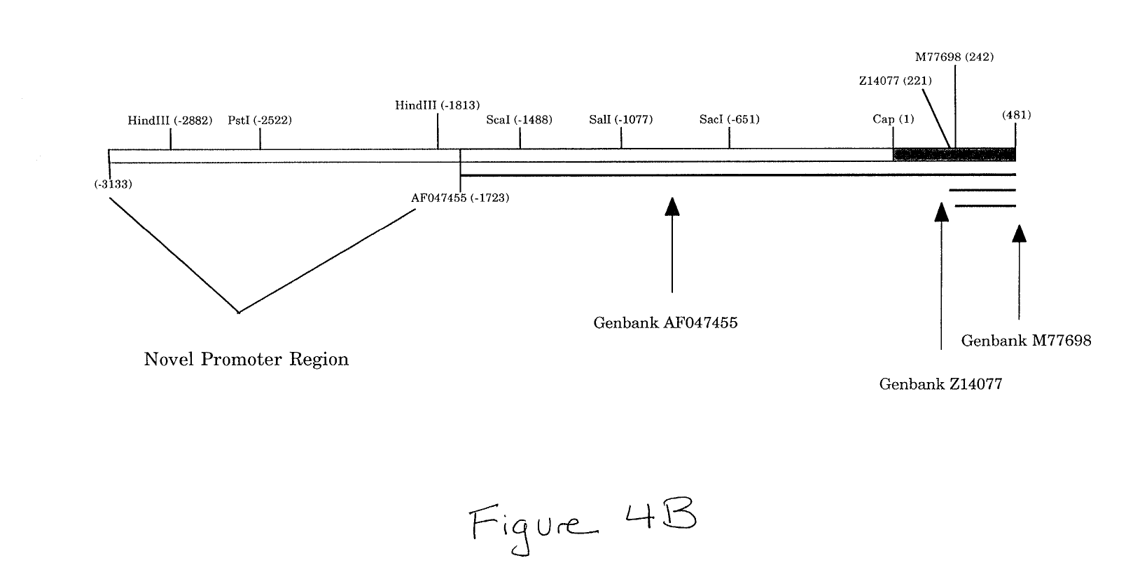

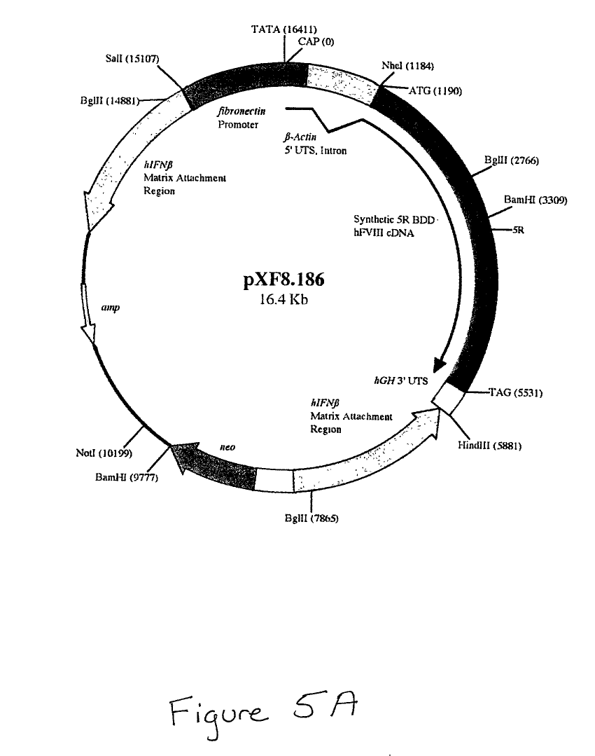

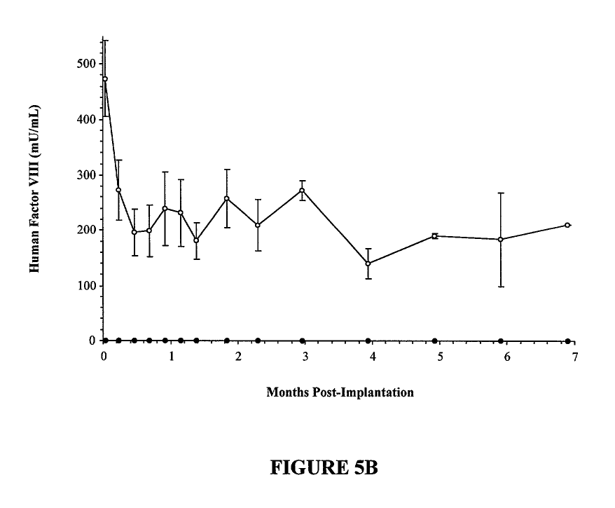

An hFVIII expression plasmid, pXF8.941 (FIG. 5A) contains a promoter region from the human γ-actin gene. The plasmid also contains a CMV immediate early I gene transcriptional enhancer inserted approximately about cap −300 bp to cap −1100 bp, or about 0.5 kb from the transcription start site. The fourth intron from the 5′ UTS of the human aldolase A gene (GenBank accession no. X12447) was inserted between the β-actin promoter region and the transcription start site.

Fibroblasts were isolated from the fascia underlying the dorsolateral dermis of New Zealand White rabbits or from human neonatal foreskins by enzymatic digestion. Fibroblasts were transfected by electroporation with 100 μg of plasmid pXF8.941. G418-resistant colonies were isolated and expanded as described essentially in Heartlein et al. (1994) Proc. of the Natl. Acad. of sci. USA 91, 10967-10971.

### Example 3

### Cell Implantations

NIH Swiss nude mice (Taconic, Germantown, N.Y.) were implanted with 5×10⁶ fibroblasts in the lesser omental recess proximal to the stomach (intraomental; 10). Briefly, animals were anesthetized using 2-2-2 tribromoethanol. The spleen was exposed and gently exteriorized. Cell slurries were injected along the axis of the spleen upon the cranial and medial aspect and within the thin membrane adjacent to the hilar surface of the spleen.

Blood was collected via the retro-orbital sinus and placed into microtainer plasma separator tubes containing lithium heparin (Becton Dickinson, Rutherford, N.J.). Platelet poor plasma was collected by centrifugation at 4° C. Plasma samples were frozen at −20° C. until analysis by hFVIII ELISA assay or immunoprecipitation as described herein.

Animal handling procedures were consistent with the recommendations of the Guide for the Care and Use of Laboratory Animals as published by the National Research Council, and were in compliance with the Animal Welfare Act.

### Example 4

### Detection of Expression

ELISA Assay for Human Factor VIII

The hFVIII ELISA is based on the use of two hFVIII-specific, non-crossreacting antibodies, e.g., monoclonal antibodies (mAb). Preferred mABs are ESH-8 and ESH-4 (described in Griffin et al. (1986) Thrombosis and Haemostasis 55:40-46; available from Scottish National Blood Transfusion Service, Edinburgh, Scotland or commercially through American Diagnostica Inc.). Briefly, sample wells, e.g., 96-well plates, are coated with a first antibody to Factor VIII (preferably ESH-8). Preferably, less than 10 μg/mL (more preferably less than 5, 4, 3, 2 or 1 μg/mL) of antibody is used to coat the plates. In this example, wells were coated with 1.0 μg/mL ESH-8 mAb and blocked with standard blocking buffer, e.g., a BSA based blocking buffer. 0.1 mL serum samples were added to each well and incubated at room temperature for about one hour. Sample wells were then washed in standard wash buffer and then incubated with horseradish peroxidase (HRP)-conjugated ESH-4. Sample wells were washed and 3,3′,5,5′-tetramethylbenzidine (TMB) substrate solution (BioRad, Inc. Hercules, California) was added. The reaction was terminated by acid addition and absorption was measured at 450 nm. The resultant absorbance values were standardized using predetermined quantities of hFVIII-containing pooled normal plasma.

Throughout the assay, the reaction mixture can include 20% plasma, for example, by adding non cross-reacting, FVIII-free plasma to maintain 20% plasma in sample dilutions beyond 1:5. In a mouse assay, this is simple, since the assay does not pick up mouse FVIII. To assay human samples, plasma from hemophiliac patients can be used. It is important to test such plasma on the assay, for certain defects leading to hemophilia A may generate an inactive FVIII molecule that can be detected using the assay. Other defects generate no recognizable FVIII by ELISA.

In addition, EDTA can be included in the wash buffer and sample buffer as a chelating agent, which it is believed can reduce the Mg2+-dependent interaction of heavy and light chains of FVIII (although the inventors do not wish to be bound by such theory). Since this ELISA assay measures light chain, it is important to reduce any potential interference. In addition, the EDTA may help to break up vWF (von Willibrand)/FVIII complexes (vWF being the “carrier” molecule for FVIII in the circulation).

The assay for factor VIII described herein can detect levels of factor VIII as low as 3.125 mU/mL. The sensitivity of this assay is thus much higher than other assays known in the art (see Hornsey et al. (1992) Transfusion Med. 2:223-229).

hFVIII Activity Assays

A) Activated Partial Thromboplastin Time (aPTT)

hFVIII (partially purified from transfected human fibroblasts or purified from CHO cells (a gift from Genetics Institute, Inc.) were assayed by a one-stage clotting method using an activated partial thromboplastin time (aPTT) reagent (Helena Laboratories, Beaumont, Tex.) and Factor VIII deficient human plasma (George King Biomedical, Overland Park, Kans.). The assay was calibrated over a range of 0.005 to 0.1 U/mL using pooled normal human plasma (NHP) (George King Biomedical), with 1 Unit of HFVIII defined as the activity present in 1 mL of NHP.

B) Coatest.®

The Coatest® Factor VIII assay kit (Chromogenix AB, Molndal, Sweden) measures the ability of hFVIII to act as a cofactor in the conversion of Factor X to Factor Xa by Factor IX in the presence of phospholipid and calcium. Factor Xa is subsequently detected by its ability to cleave a chromogenic substrate. A standard curve was prepared from dilutions of pooled normal human plasma (George King Biomedical, Overland Park, Kans.). The assay was linear using a log-log fit of the data over a range of 142 to 1000 mU/mL. The assay was performed essentially as described by the manufacturer.

C) SDS-PAGE/Western and Thrombin Digestion

Partially purified hFVIII derived from transfected human fibroblasts was diluted with 20 mM imidazole, pH 6.9, containing 137 mM NaCl, 2.5 mM CaCl2, and 0.1% Tween 20. The samples were digested with thrombin at a 1:10 ratio of thrombin units to hFVIII units. Samples were taken at various time points and the reactions were stopped by adding SDS-PAGE sample buffer containing 0.1 M DTT. The thrombin fragmentation patterns of the samples were analyzed by SDS-PAGE and Western blotting.

D) Immunoprecipitation.

Plasma samples (100 μL) were incubated with Affi-gel 10 beads (BioRad, Inc. Hercules, Calif.) containing bound anti-hFVIII antibody. The antibody used in these studies was SAF8C-Ig, a sheep anti-hFVIII polyclonal antibody (Enzyme Research Laboratories, South Bend, Ind.). Samples were run on an 8-16% Tris-glycine gel and transferred to nitrocellulose. The blot was incubated with sheep anti-hFVIII-HRP antibody overnight.

E) Immunohistochemistry

Tissues were preserved in 10% neutral buffered formalin and embedded in paraffin. Human FVIII was detected in tissue sections (5 μm) using a sheep anti-human FVIII antibody (Accurate Chemical, Westbury, N.Y.) followed by a biotinylated donkey anti-sheep IgG (Accurate Chemical, Westbury, N.Y.). HRP-linked to aviden (ABC, Vector Laboratories, Burlingame, Calif.) served as an enzymatic tag, and color was developed using diaminobenzidine. Negative control slides were stained with sheep anti-human IgG substituting for the primary antibody.

### Example 5

### Implantation of hFVIII-Producing Fibroblasts and Analysis of In Vivo Expression

A human fibroblast clone producing HFVIII was implanted into five male nude mice (FIG. 5B). Five additional mice served as controls. Sustained delivery of hFVIII was found with human fibroblasts implanted into nude mice beginning with the stabilization of plasma hFVIII levels during the first week following implantation. Human FVIII was detected in plasma for seven months at mean levels of approximately 200 mU/mL. No significant hFVIII was detected in control animals.

To confirm that hFVIII antigen detectable by the ELISA assay corresponded to a plasma protein of the size predicted for hFVIII, Western blot analysis was performed on selected samples from mice. Fresh plasma samples drawn from the mice were immunoprecipitated using an anti-hFVIII antibody and subjected to SDS-PAGE and Western blotting. Results show that plasma from mice implanted with hFVIII expressing cells has clear anti-hFVIII reactive bands at sizes predicted for hFVIII. These bands are not present in control mouse plasma.

The results presented in these examples demonstrate that stably transfected human cells, e.g., clonal skin fibroblasts, expressing a heterologous product, e.g., hFVIII, can lead to long-term (over 7 months in this example) delivery of the clotting factor to the systemic circulation of a subject, e.g., a mouse, a rabbit, a monkey, or a human. Plasma levels of 100-300 mU/mL were stably maintained, corresponding to 10-30% of the normal human level.

It is generally considered that increasing the level of hFVIII from <1% seen in severe hemophilia A patients to approximately 1-5% would dramatically improve the clotting phenotype of these patients. These data suggest that administration of stably transfected clonal skin fibroblasts expressing hFVIII can be used successfully for the treatment of hemophilia A by cell therapy (e.g., Transkaryotic Therapy™).

These results can be extrapolated for considering the treatment of hemophilia A patients. Because a 30 g mouse is approximately 2000 times smaller than a 70 kg patient, one might expect to require nearly 10 billion cells to treat human patients in order to achieve the 10-30% of normal hFVIII levels reported here. However, several reports describing the half-life (t_{1/2}) of different forms of hFVIII in mice reported t_{1/2} values ranging from 1-5 hours, which range is approximately 2 to 10-fold lower than the t_{1/2} reported for recombinant hFVIII in normal humans. Furthermore, the levels seen in immunocompromised mice are up to 10-fold higher than the levels needed to provide clinical benefit to hemophilia A patients (>1%). This reduced half-life suggests an implant size of approximately 100 to 500 million cells producing per cell levels similar to those presented here would be required. An implant of this size is achievable on a routine basis for clonal strains of hFVIII-expressing human fibroblasts. Finally, it is likely that autologous cells implanted in a patient will fare better than the xenogenic implantations described here.

Unlike the immunogenicity of gene products known to plague most viral vectors used in gene therapy, the simple plasmid-based system used here coupled with the use of clonal, autologous cells effectively reduces the chance of eliciting an immune response and minimizes the risk of a tumorigenic event. In contrast to widely used in vivo delivery systems for delivery of hFVIII where viral vectors infect millions of cells randomly throughout multiple organs of the body, the system described here has the added benefit of being confined to a single genetic modification of the clonal strain and a single implant site for stable engraftment. If necessary during the course of treatment, the implant could be surgically excised.

The system described here is designed to improve the clinical outcome of hemophilia A patients by providing a stable level of HFVIII in the blood. Moreover, treatment of hemophilia A patients with their own fibroblasts transfected, cloned, and selected to stably produce hFVIII promises to free these patients from the substantial risks and costs of frequent injections of hFVIII concentrates. A Phase I clinical trial of the system is in progress.

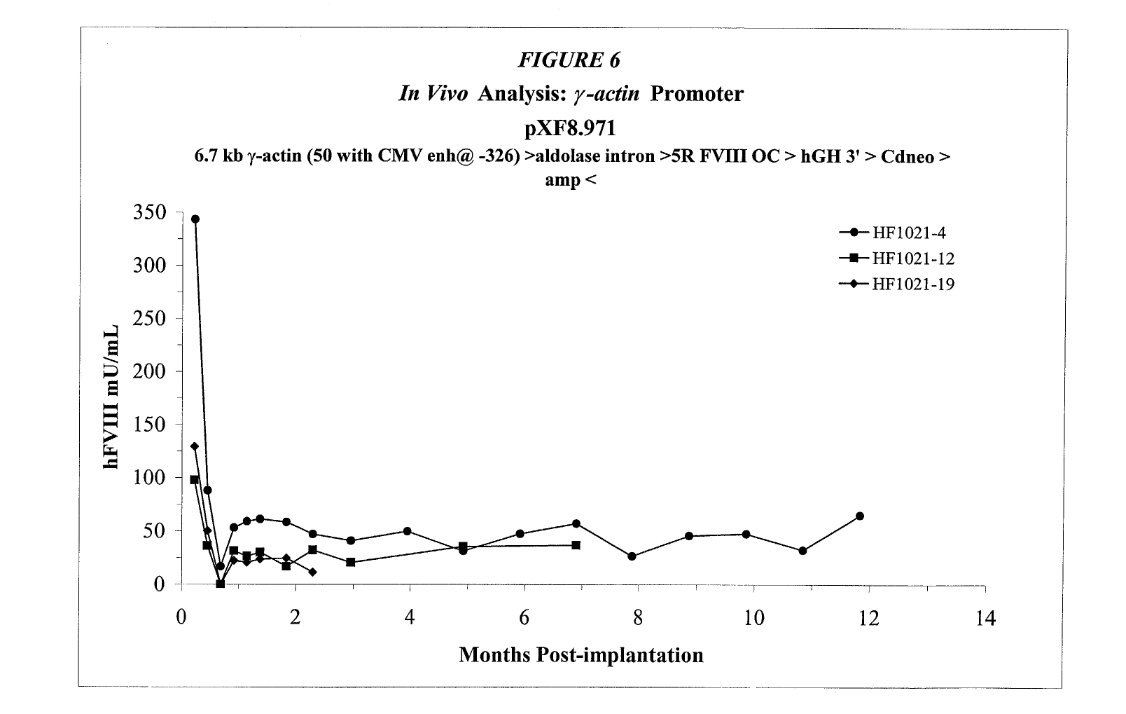

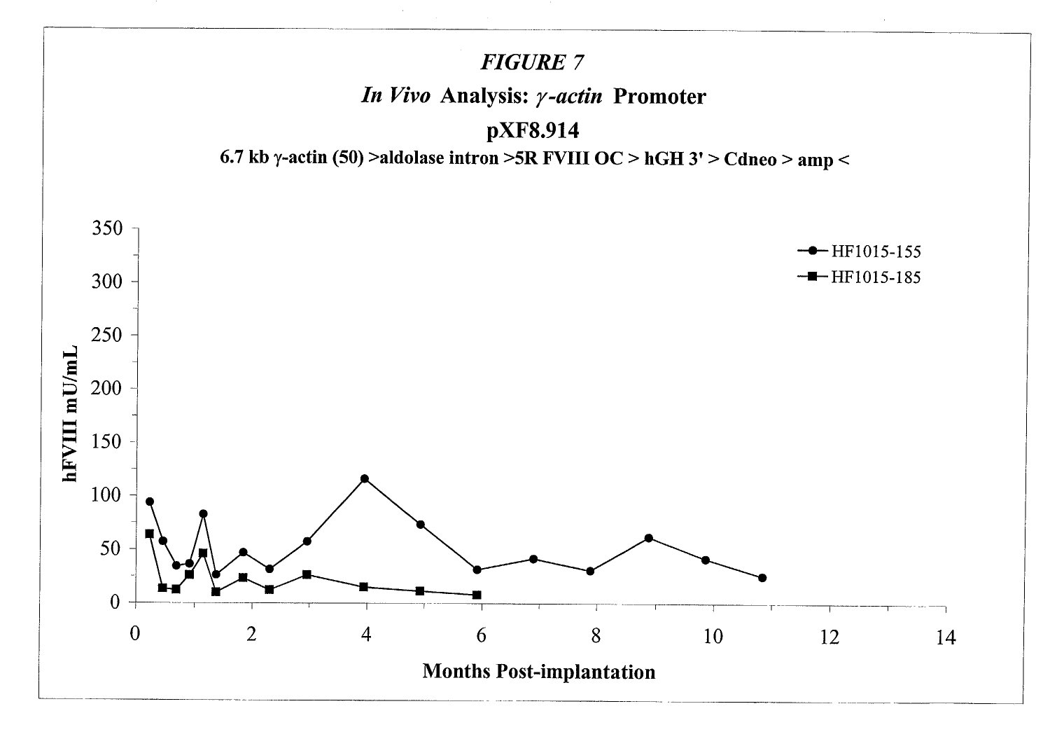

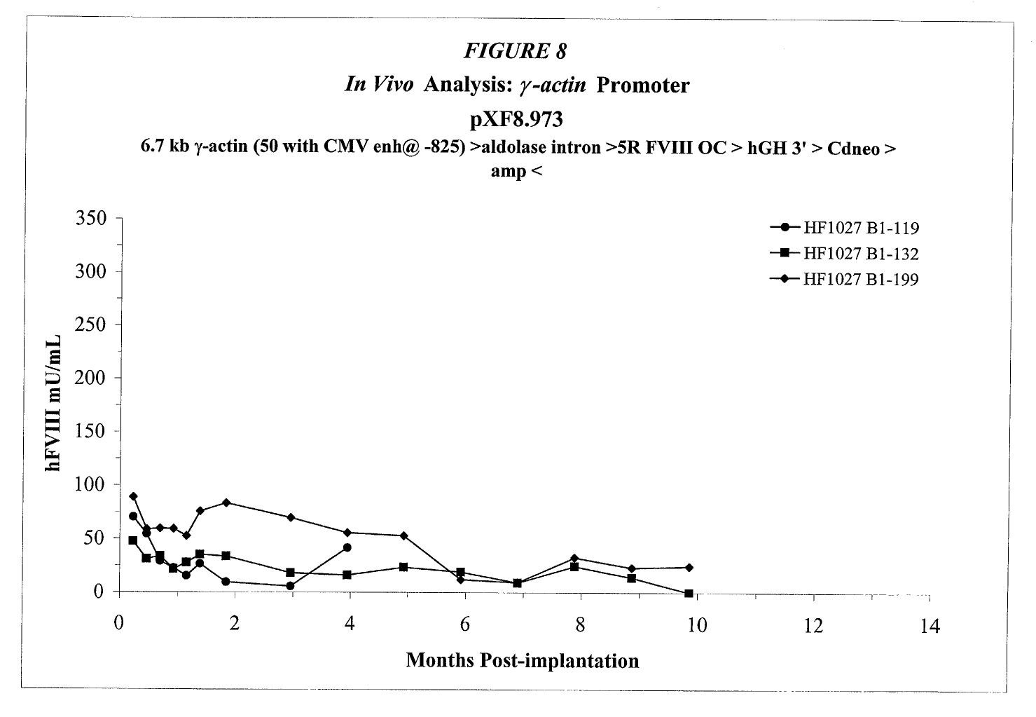

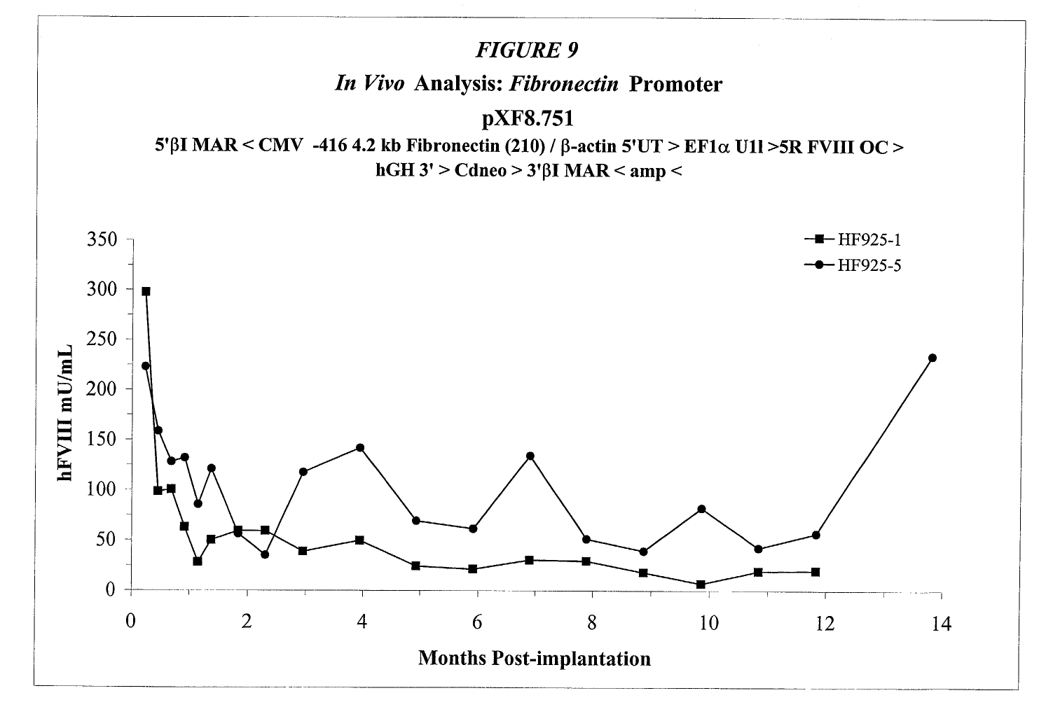

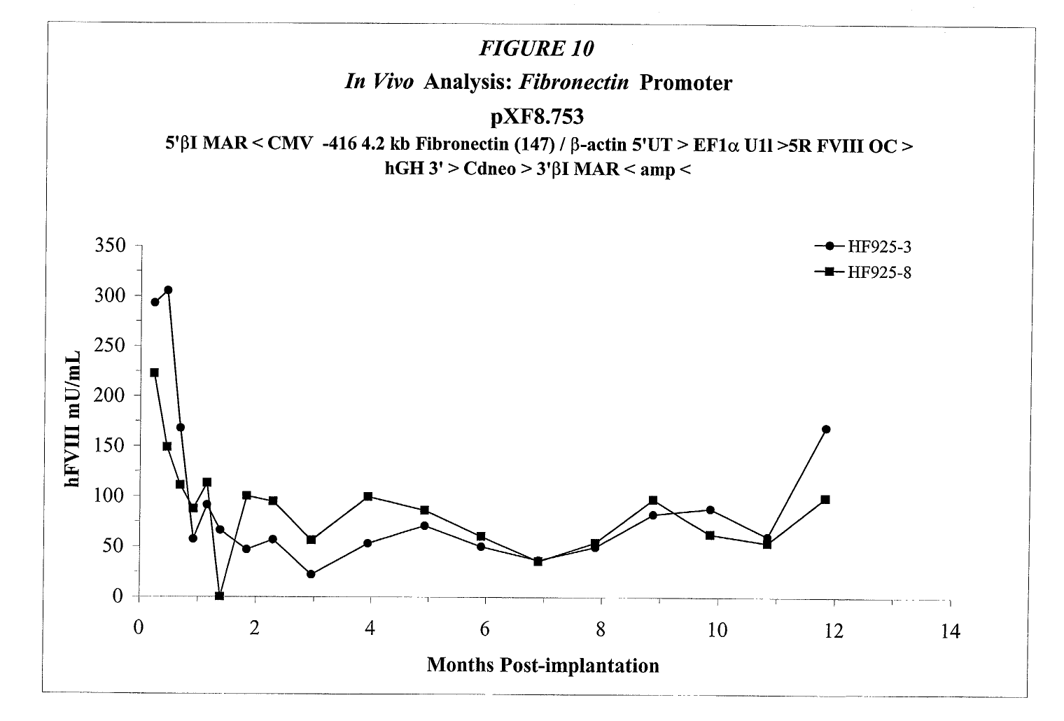

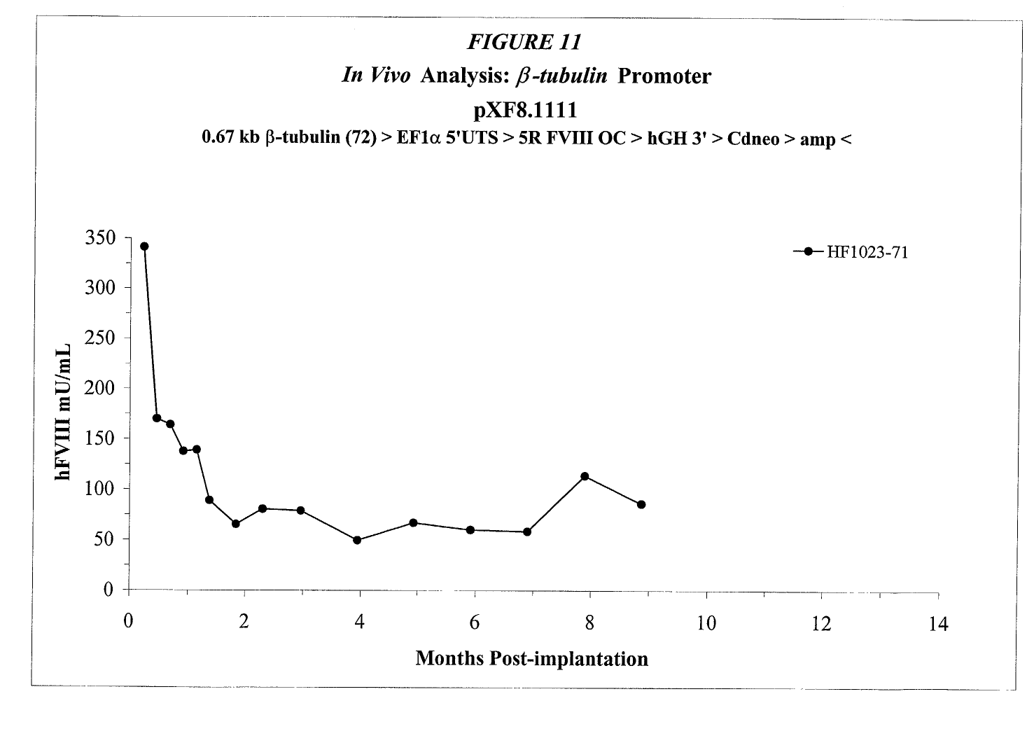

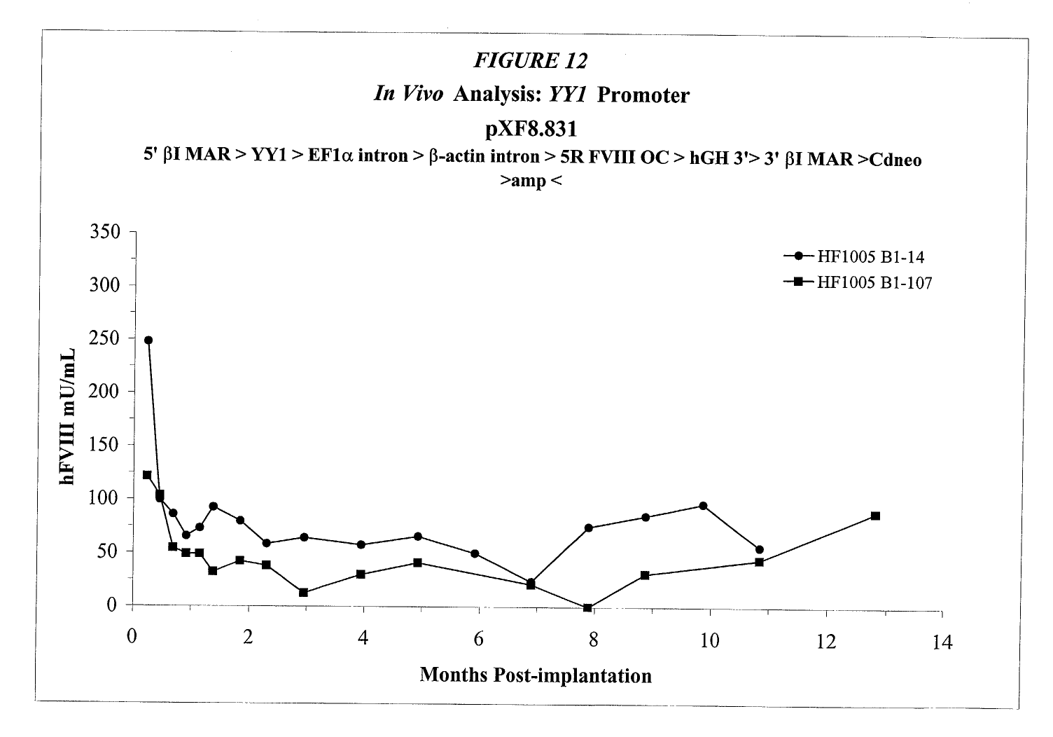

Long term in vivo expression in mice from clones generated by transfection with other exemplary constructs of the invention is shown in FIGS. 6-12, as follows: construct comprising a γ-actin promoter region (FIGS. 6-8); construct comprising a fibronectin promoter region (FIGS. 9-10); construct comprising a β-tubulin promoter region (FIG. 11); and a construct comprising a YY1 promoter region (FIG. 12).

These examples demonstrate that long-term delivery of therapeutic levels of a product, e.g., hFVIII, to the bloodstream can be achieved by implantation of clonal normal cells, e.g., fibroblasts, stably transfected with a construct described herein, e.g., a hFVIII expression plasmid as described herein.

All references and patents cited herein are incorporated by reference in their entirety. A number of embodiments of the invention have been described. Nevertheless, it will be understood that various modifications may be made without departing from the spirit and scope of the invention. Accordingly, other embodiments are within the scope of the following claims.

What is claimed is:

1. An isolated nucleic acid molecule comprising the nucleotide sequence of SEQ ID NO: 1, SEQ ID NO:2, SEQ ID NO:3, or SEQ ID NO:4.

2. An isolated nucleic acid sequence which is at least 60% identical to the nucleotide sequence of SEQ ID NO:1, SEQ ID NO:2, SEQ ID NO:3, or SEQ ID NO:4.

3. The isolated nucleic acid molecule of claim 1 further comprising a vector nucleic acid sequence.

4. The nucleic acid molecule of claim 3 further comprising a nucleic acid sequence encoding a heterologous polypeptide.

5. A host cell which contains the nucleic acid molecule of claim 1, 2, 3, or 4.

6. The host cell of claim 5 which is a mammalian cell.

7. The host cell of claim 6 which is a human fibroblast.

8. A construct suitable for expression in a mammalian cell, comprising a human γ-actin promoter region operably linked to a heterologous nucleic acid sequence.

9. The construct of claim 8, further comprising at least one of the following elements:
(a) an enhancer;
(b) a heterologous 5′ UTS,
(c) a β-actin 5′ UTS fusion; and
(d) a MAR, preferably a βI MAR.

10. The construct of claim 8, further comprising at least one of the following elements:
(a) a CMV enhancer;
(b) a 5′ UTS selected from: an aldolase 5′ UTS, an EF1-α 5′ UTS, or a β-actin 5′ UTS;
(c) a γ-actin 5′ UTS fusion; and
(d) a βI MAR.

11. The construct of claim 8, further comprising at least one of the following elements:
(a) a CMV enhancer placed between cap −300 bp to cap −1100 bp;
(b) an aldolase 5′ UTS;
(c) a γ-actin 5′ UTS fusion of cap +25 to cap +50 bp in length; and
(d) a βI MAR.

12. The construct of claim 8, further comprising a CMV enhancer at cap −825 bp, and a γ-actin 5′ UTS fusion of cap +25 base pairs in length fused to an aldolase 5′ UTS.

13. The construct of claim 8, wherein said γ-actin promoter region includes at least 2 contiguous nucleotides from the sequence of SEQ ID NO: 1.

14. The construct of claim 8, wherein said γ-actin promoter region includes at least 3000 contiguous nucleotides from the sequence of SEQ ID NO: 1.

15. The construct of claim 8, wherein the γ-actin promoter region is between 0.9 kb and 7.2 kb in length.

16. The construct of claim 15, wherein the γ-actin promoter region is between 3.6 kb and 6.5 kb.

17. The construct of claim 13 further comprising at least one of the following elements:
(a) an enhancer;
(b) a heterologous 5′ UTS;
(c) a γ-actin 5′ UTS fusion;
(d) a MAR.

18. The construct of claim 13 further comprising at least one of the following elements:
(a) a CMV enhancer;
(b) a 5′ UTS selected from: an aldolase 5′ UTS, an EF1-α 5′ UTS, or β-actin 5′ UTS;
(c) a γ-actin 5′ UTS fusion of cap +25 to cap +50 bp in length; and
(d) a βI MAR.

19. The construct of claim 13 further comprising at least one of the following elements:
(a) a CMV enhancer placed between cap −300 to cap −1100 bp;
(e) an aldolase 5′ UTS;
(f) a γ-actin 5′ UTS fusion of cap +25 to cap +50 bp in length; and
(g) a βI MAR.

20. The construct of claim 13 further comprising a CMV enhancer at cap −825 bp, and a γ-actin 5′ UTS fusion of cap +25 base pairs in length fused to an aldolase 5′ UTS.

21. The construct of claim 8, wherein the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, galactosidase, N-acetylgalactosamine-4-sulfatase (arylsulfatase B), β-glucuronidase or a biologically active fragment thereof.

22. A construct suitable for expression in a human cell comprising a human β-tubulin promoter region operably linked to a heterologous nucleic acid sequence.

23. The construct of claim 22, further comprising at least one of the following elements:
(a) a heterologous 5′ UTS;
(b) an enhancer; and
(c) a U1 intron.

24. The construct of claim 22, further comprising at least one of the following elements:
(a) a 5′ UTS selected from: an aldolase 5′ UTS, an EF1-α 5′ UTS, and a β-actin 5′ UTS;
(b) a CMV enhancer;
(c) a U1 intron selected from: a GAPDH U1 intron and a β-actin U1 intron.

25. The construct of claim 22, further comprising an EF1-α 5′ UTS.

26. The construct of claim 22, wherein said β-tubulin promoter region includes at least two contiguous nucleotides from the sequence of SEQ ID NO:3.

27. The construct of claim 22, wherein said β-tubulin promoter region includes at least 1000 contiguous nucleotides from the sequence of SEQ ID NO:3.

28. The construct of claim 22, wherein the β-tubulin promoter region is between 0.6 kb and 12 kb in length.

29. The construct of claim 22, wherein the β-tubulin promoter region is between 0.6 kb and 2.3 kb.

30. The construct of claim 26, further comprising at least one of the following elements:
(a) a heterologous 5′ UTS;
(b) an enhancer; and
(c) a U1 intron.

31. The construct of claim 26, further comprising at least one of the following elements:
(a) a 5′ UTS selected from: an aldolase 5′ UTS, an EF1-α 5′ UTS, and a β-actin 5′ UTS;
(b) a CMV enhancer;
(c) a U1 intron selected from the group of: a GAPDH U1 intron, and a β-actin U1 intron.

32. The construct of claim 26, further comprising an EF1-α 5′ UTS.

33. The construct of claim 22, wherein the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, β-galactosidase, N-acetylgalactosamine-4-sulfatase (arylsulfatase B), β-glucuronidase or a biologically active fragment thereof.

34. A construct suitable for expression in a human cell, comprising a human YY1 promoter region operably linked to a heterologous nucleic acid.

35. The construct of claim 34, further comprising at least one of the following elements:
(a) an enhancer;
(b) a heterologous 5′ UTS; or
(c) a MAR.

36. The construct of claim 34, further comprising at least two of:
(a) a CMV enhancer;
(b) at least one heterologous 5′ UTS selected from the group of: an aldolase 5′ UTS, an EF1-α 5′ UTS and a β-actin 5′ UTS; or
(c) a βI MAR.

37. The construct of claim 34, further comprising:
(a) a CMV enhancer placed between cap −100 to cap −1000 bp;
(b) an EF1-α 5′ UTS fused to a β-actin 5′ UTS; and
(c) a βI MAR

38. The construct of claim 34, wherein said YY1 promoter region includes at least two contiguous nucleotides from the sequence of SEQ ID NO:4.

39. The construct of claim 34, wherein said YY1 promoter region is between 1.7 kb and 3.1 kb in length.

40. The construct of claim 38, further comprising at least one of the following elements:
(a) an enhancer;
(b) a heterologous 5′ UTS; or
(c) a MAR.

41. The construct of claim 38, further comprising at least two of:
(a) a CMV enhancer;
(b) at least one heterologous 5′ UTS selected from the group of: an EF1-α 5′ UTS, an aldolase 5′ UTS and a β-actin 5′ UTS; or
(c) a βI MAR.

42. The construct of claim 38, further comprising:
(a) a CMV enhancer placed between cap −100 to cap −1000 base pairs;
(b) an EF1-α 5′ UTS fused to a β-actin 5′ UTS; and
(c) a βI MAR

43. The construct of claim 34, wherein the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, galactosidase, N-acetylgalactosamine-4-sulfatase (arylsulfatase B), β-glucuronidase or a biologically active fragment thereof.

44. A construct suitable for expression in a human cell, comprising a human fibronectin promoter region operably linked to a heterologous nucleic acid sequence, provided that said promoter region includes at least two contiguous nucleotides from SEQ ID NO:2.

45. A construct suitable for expression in a human cell, comprising a human fibronectin promoter region operably linked to a heterologous nucleic acid sequence, provided that said promoter region includes at least one of the following elements:
(a) an enhancer;
(b) a U1 intron.

46. The construct of claim 44, wherein the construct includes at least two of the following elements:
(a) an enhancer;
(b) a MAR;
(c) a U1 intron;
(d) a 5′ UTS; or
(e) a fibronectin 5′ UTS fusion.

47. The construct of claim 44, wherein the construct comprises at least two of the following elements:
(a) a CMV enhancer;
(b) a βI MAR;
(c) a U1 intron selected from the group of: an EF1-α, aldolase, and GAPDH U1 intron;
(d) a 5′ UTS selected from the group of: a β-actin, aldolase, and EF1-α 5′ UTS;
(e) a fibronectin 5′ UTS fusion of between cap +147 to cap +270 bp in length.

48. The construct of claim 44, wherein the construct comprises a β-actin 5′ UTS fused to an EF1-α U1 intron and at least one of the following elements:
(a) a CMV enhancer placed between cap −50 to cap −700 bp;
(b) a βI MAR; or
(c) a fibronectin 5′ UTS fusion of between cap +147 to cap +270 bp in length.

49. The construct of claim 44, wherein the human fibronectin promoter region is between 0.5 kb and 8.8 kb in length.

50. The construct of claim 49, further compromising at least one of the following elements:
(a) an enhancer;
(b) a MAR;
(c) a U1 intron;
(d) a 5′ UTS;
(e) a U1 intron in combination with a 5′ UTS; or
(f) a fibronectin 5′ UTS fusion.

51. The construct of claim 49, wherein the construct includes at least one of the following elements:
(a) a CMV enhancer;
(b) a βI MAR;
(c) a U1 intron selected from the group of: an EF1-α aldolase, or GAPDH U1 intron;
(d) a 5′ UTS selected from the group of: β-actin, aldolase, or EF1-α 5′ UTS;
(e) a fibronectin 5′ UTS fusion of between cap +147 to cap +270 bp in length.

52. The construct of claim 49, wherein the construct comprises a β-actin 5′ UTS fused to an EF1-α U1 intron and at least one of the following elements:
(a) a CMV enhancer placed between cap −50 to cap −700 bp;
(b) a βPI MAR; or
(c) a fibronectin 5′ UTS fusion of between cap +147 to cap +270 bp in length.

53. The construct of claim 44, wherein the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, β-galactosidase, N-acetylgalactosamine-4-sulfatase (arylsulfatase B), β-glucuronidase or a biologically active fragment thereof.

54. A construct suitable for expression in a human cell, comprising a human β-actin promoter region operably linked to a heterologous nucleic acid sequence, provided that said promoter region includes at least one of the following elements:
(a) an MAR;
(b) a β-actin 5′ flank addition; or
(c) a β-actin 5′ UTS fusion.

55. A construct suitable for expression in a human cell, comprising a human β-actin promoter region operably linked to a heterologous nucleic acid sequence, wherein the construct includes at least one of the following elements:
(a) an MAR;
(b) an enhancer;
(c) a heterologous 5′ UTS;
(d) a U1 intron; or
(e) a β-actin 5′ UTS fusion;
provided that if the construct includes (b) it does not include (c) and if the construct includes (c) it does not include (b).

56. The construct of claim 54, wherein the construct comprises at least two of the following elements:
(a) a βI MAR;
(b) a CMV-based enhancer;
(c) an aldolase 5′ UTS;
(e) a β-actin 5′ UTS fusion of cap +77 bp in length; or
(f) an EF1-α U1 intron.

57. The construct of claim 54, wherein the construct comprises at least two of the following elements:
(a) a βI MAR;
(b) a CMV-based enhancer selected from the group of: a CMV enhancer and a 4 tandem 53 bp repeat element derived from a CMV enhancer, wherein the enhancer is placed between cap −50 and cap −500 bp;
(c) an aldolase 5′ UTS;
(d) a 5′ flank addition of up to 2.5 kb;
(e) a β-actin 5′ UTS fusion of cap +77 bp in length; or
(f) an EF1-α U1 intron.

58. The construct of claim 54, wherein the heterologous nucleic acid is a nucleic acid encoding a Factor VIII, Factor IX, human growth hormone (hGH), erythropoietin (EPO), glucagon-like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, β-galactosidase, N-acetylgalactosamine-4-sulfatase (arylsulfatase B), β-glucuronidase or a biologically active fragment thereof.

59. A cell transfected with a construct described herein.

60. The cell of claim 59, wherein the cell is a human cell.

61. The cell of claim 60, wherein the human cell is a fibroblast.

62. A method of producing a substance, comprising:
providing a human cell which includes a construct described herein; and
allowing said cell to express a heterologous polypeptide, thereby producing a substance.

63. The method of claim 62, wherein the human cell is a human fibroblast.

64. A method of supplying a substance to a subject, comprising:
(a) providing a human cell which includes a construct described herein; and
(b) allowing said cell to express a heterologous polypeptide; and
(c) administering said heterologous polypeptide to the subject, thereby supplying a substance to a subject.

65. The method of claim 64, wherein the human cell is a human fibroblast.

66. A method of treating a disorder in a subject, comprising:
providing a human cell which includes a construct described herein; and
allowing said cell to express a heterologous polypeptide in vivo in the subject, thereby treating a disorder in a subject.

67. The method of claim 66, wherein the human cell is a human fibroblast.

68. The method of claim 62, 64, or 66, wherein the heterologous protein is expressed in vitro.

69. The method of claim 62, 64, or 66, wherein the heterologous protein is expressed in vivo.

70. The method of claim 62, 64, or 66, wherein the cell ca n be an autologous, allogeneic, or xenogeneic cell.

71. A method of providing a heterologous protein to a subject, comprising:
(a) providing a human cell which includes a construct described herein; and
(b) allowing said cell to produce the heterologous protein in vivo in said subject, thereby providing a heterologous protein to a subject.

72. The method of claim 71, wherein the cell can be an autologous, allogeneic, or xenogeneic cell.

73. A method of treating a disorder in a subject, comprising:
identifying a subject in need of a product; and
introducing into the subject a construct described herein, wherein the construct causes the production of the product in an amount sufficient to ameliorate a symptom of said disorder;
thereby treating the disorder in the subject.

74. The method of claim 73, wherein the product is Factor VIII and wherein the method further comprises evaluating the level of Factor VIII in a blood sample of the subject (blood includes whole blood, cells, serum or plasma), the evaluating step comprising:
(a) contacting a blood sample of the subject with a first antibody to Factor VIII thereby forming a reaction mixture;
(b) thereafter contacting the reaction mixture with a labeled second antibody to Factor VIII; and
(c) detecting the amount of label in the reaction mixture, the amount of label being correlated to the amount of Factor VIII in the sample.

75. The method of claim 74, wherein less than 6.25 mU/mL of Factor VIII can be detected.

76. The method of claim 74, wherein the first antibody is ESH-8.

77. The method of claim 74, wherein the second labeled antibody is labeled ESH-4.

78. A method of evaluating the level of Factor VIII in a blood sample of the subject, comprising:
(a) contacting a blood sample of the subject with a first antibody to Factor VIII thereby forming a reaction mixture;
(b) thereafter contacting the reaction mixture with a second labeled antibody to Factor VIII;
(c) detecting the amount of label in the reaction mixture, the amount of label being correlated to the amount of Factor VIII in the sample,
thereby evaluating the level of Factor VIII in a blood sample.

79. The method of claim 78, wherein less than 6.25 mU/mL of Factor VIII can be detected.

80. The method of claim 78, wherein less than 5 mU/mL of Factor VIII can be detected.

81. The method of claim 78, wherein less than 4 mU/mL of Factor VIII can be detected.

82. The method of claim 78, wherein the first antibody is ESH-8.

83. The method of claim 78, wherein the first antibody is used at a concentration of less than 5 μg/mL.

84. The method of claim 78, wherein the first antibody is used at a concentration of less than 3 μg/mL.

85. The method of claim 78, wherein the blood sample is less than 100 μL.

86. The method of claim 78, wherein the second labeled antibody is labeled ESH-4.

87. A kit for the detection of Factor VIII, comprising:
(a) a first antibody to Factor VIII;
(b) a second labeled antibody to Factor VIII; and
(c) instructions for using the first and second antibodies to detect Factor VIII at a concentration of 3.5 mU/mL or more.
